(19) United States
(12) Patent Application Publication
(10) Pub. No.: US 2021/0246454 A1
(43) Pub. Date: Aug. 12, 2021

(54) SPLICE MODULATING OLIGONUCLEOTIDES TARGETING RECEPTOR FOR ADVANCED GLYCATION END PRODUCTS AND METHODS OF USE

(71) Applicant: LifeSplice Pharma LLC, Malvern, PA (US)

(72) Inventors: Nicole M. LYKENS, Malvern, PA (US); Gordon J. LUTZ, Malvern, PA (US); Melanie K. TALLENT, Malvern, PA (US)

(21) Appl. No.: 17/056,368

(22) PCT Filed: May 17, 2019
(86) PCT No.: PCT/US2019/032974

§ 371 Date: Nov. 17, 2020

Related U.S. Application Data
(60) Provisional application No. 62/672,655, filed on May 17, 2018.

(51) Int. Cl. C12N 15/113; A01K 67/027; A61K 49/00

(52) U.S. Cl. CPC C12N 15/1138; A01K 67/0278; A01K 2267/03; A01K 2207/15; A01K 2227/105; A61K 49/0008

(57) ABSTRACT

The invention provides splice modulating oligonucleotides (SMOs) designed to modulate the splicing of a RAGE pre-mRNA, compositions including the SMOs, and methods of treating and preventing diseases and conditions using the SMOs and compositions.

### STATEMENT AS TO FEDERALLY FUNDED RESEARCH

This invention was made with government support under Grant No. 1R21AG060208-01 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on May 17, 2019 is named “50972-003WO2_Sequence_Listing_05.17.2019_ST25” and is 568.562 bytes in size.

### BACKGROUND

The human Receptor for Advanced Glycation End products (RAGE; AGER: HGNC #320) is a member of the immunoglobulin superfamily of receptors and is expressed on a wide array of cell types (Gray et al., Nuc. Acids Res. 41:D545-D552, 2013). In addition to being processed to form mRNA encoding full-length RAGE protein (flRAGE), RAGE pre-mRNA is extensively alternatively spliced, yielding splice variant mRNAs encoding RAGE proteins with altered amino acid compositions of the ligand binding domain or removal of the transmembrane region, with the latter class of variants encoding secreted, non-membrane bound forms of the receptor (sRAGE, e.g., RAGEv1; Park et al., Mol. Immunol. 40(16):1203-1211, 2004; Schlueter et al., Biochim. Biophys. Acta, 1630 (1):1-6, 2003; Yonekura et al., Biochem. J. 370(Pt 3):1097-1109, 2003).

RAGE recognizes 3-dimensional structures rather than specific amino acid sequences, providing for interactions with a diverse repertoire of ligands including, e.g., advanced glycation end products (AGEs), S100/calgranulins, high-mobility group box 1 (HMGB1), amyloid-β peptides (Hiwatashi et al., Ann. Surg. Oncol. 15(3):923-933, 2008), and MAC-1 (leukocyte integrin ITGAM; Chavakis et al., J. Exp. Med. 198(10):1507-1515, 2003; Yan et al., Expert. Rev. Mol. Med. 11 e9, 2009). Activation of RAGE affects several important signaling pathways which, in some instances, may target central transcription factors to regulate gene expression and/or play roles in immune regulation (see, e.g., Mahajan et al., Int. J. Cardiol., 2013). Accordingly, dysregulation (e.g., over-activation) of RAGE signaling is associated with a wide variety of diseases and conditions including, e.g., neurodegenerative, metabolic, cardiovascular, immunological, autoimmune, liver, and lung diseases, as well as cancer.

AGEs are RAGE ligands that are undesirable metabolic by-products from non-enzymatic glycoxidation of proteins and lipids (e.g., from natural aging, hyperglycemia, oxidative stress, and renal failure). The body manages AGEs through a natural clearance mechanism (e.g., binding to RAGEv1), but AGEs accumulate over time in a variety of tissues and are associated with changes in tissue/cell properties and organ dysfunction (Basta et al., Cardiovasc. Res. 63(4):582-592, 2004). In certain disease states, excessive AGE generation can overwhelm the clearance mechanisms, resulting in over-activating of RAGE, which leads to leading to damage to cells and organs. For example, binding of ligands (such as AGEs) to membrane bound RAGE activates damaging cellular responses including inflammatory signaling, transcriptional dysregulation, and oncogenic signaling. In contrast, sRAGE isoforms (e.g., RAGEv1) act as extracellular decoys by binding AGEs and other flRAGE ligands, greatly reducing their availability to trigger the aforementioned pathogenic signaling processes transduced by activated flRAGE. In addition to its decoy function to reduce flRAGE signaling, sRAGE (in particular synthetic sRAGE, or syn-sRAGE) was found to decrease the chronic inflammatory pain delayed hypersensitivity response in both wild type (WT) and RAGE knockout (KO) mice, indicating that sRAGE has effects via a mechanism that does not involve reduced membrane-bound RAGE signaling (Liliensiek et al., J. Clin. Invest., 113(11):1641-1650, 2004).

The diversity of diseases and conditions associated with RAGE (e.g., RAGE over-activation) makes RAGE a desirable therapeutic target.

### SUMMARY

The invention provides compositions and methods for treating a subject at risk of, susceptible to, or having a disease, disorder, or condition associated with RAGE mRNA expression or RAGE protein expression or function. In one embodiment, a RAGE mRNA may be an alternatively spliced, aberrantly spliced, overexpressed, or unwanted mRNA (e.g., a RAGE mRNA comprising the full length receptor or a membrane-bound isoform that encodes a protein that results in, causes, produces, or pre-disposes a subject to a disease or disorder). In another embodiment, splicing of a RAGE pre-mRNA is not a cause of a disease or disorder, but modulation of the splicing of the RAGE pre-mRNA reduces at least one symptom of the disease or disorder. In another embodiment, the invention provides methods of preventing or treating in a subject, a disease, disorder, or condition associated with RAGE pre-mRNA splicing, the methods comprising administering to the subject an SMO or composition described herein, or a vector or transgene encoding the same.

Accordingly, certain embodiments of the invention provide methods of treating or preventing a disease, disorder or condition in subject (e.g., a mammal, such as a human), comprising administering an SMO or composition described herein to the subject.

In certain embodiments, the SMO administration reduces expression of RAGE isoforms which have receptor signaling function. In certain embodiments, the SMO specifically binds to a RAGE pre-mRNA sequence, wherein when the SMO specifically binds to the RAGE pre-mRNA sequence, exon 9, intron 9, exon 10, or any combination thereof, in the resulting RAGE mRNA, and wherein the resulting mRNA encodes a RAGE protein. In certain embodiments, the RAGE protein has decoy receptor function.

Certain embodiments of the invention provide an SMO as described herein for the prophylactic or therapeutic treatment of a disease or disorder in a subject. Certain embodiments of the invention provide the use of an SMO as described herein to prepare a medicament for treating a disease or disorder in a subject. Certain embodiments of the invention provide an SMO as described herein for use in medical therapy. Certain embodiments of the invention provide an SMO as described herein for use in treating a disease or disorder.

The invention thus provides methods of modulating splicing of a Receptor for Advanced Glycation End products (RAGE) pre-mRNA. The methods include contacting a plurality of cells with a splice modulating oligonucleotide (SMO) that specifically binds to a complementary sequence of a pre-mRNA that undergoes splicing to form mRNA encoding a RAGE protein, wherein the SMO alters the relative amounts of mRNA encoding soluble and membrane bound isoforms of RAGE protein produced by the pre-mRNA splicing.

In some embodiments, the SMO increases the amount of mRNA encoding a soluble isoform of RAGE protein produced.

In some embodiments, the SMO decreases the amount of mRNA encoding a membrane bound isoform of RAGE protein.

In some embodiments, the SMO directs read-through of the 5′ splice site of exon 9 of the RAGE pre-mRNA, resulting in inclusion of part or all of intron 9, or exclusion of exon 10, or any combination thereof, in the RAGE pre-mRNA.

In some embodiments, the plurality of cells is in vitro, while in other embodiments plurality of cells is in vivo.

In some embodiments, the SMO specifically binds to a complementary sequence of RAGE pre-mRNA in at least one of the group consisting of an exon, an intron, a 5′ UTR, a 3′ UTR, a splice junction, an exon:exon splice junction, an exonic splicing silencer (ESS), an exonic splicing enhancer (ESE), an intronic splicing silencer (ISS), and/or an intronic splicing enhancer (ISE) or a combination of any of the aforementioned in the RAGE pre-mRNA.

In some embodiments, the SMO produces at least a 5 percent increase in read-through of the 5′ splice site of exon 9, resulting in inclusion of part or all of intron 9, or exclusion of exon 10, or any combination thereof, in a RAGE mRNA, as compared to baseline untreated cells, and alters expression of RAGE or one or more isoforms thereof.

In some embodiments, the plurality of cells are in vivo and the SMO is administered to a subject to treat a disease or condition selected from the group consisting of Alzheimer's disease, amyotrophic lateral sclerosis, diabetes, glucose tolerance, diabetic allodynia and neuropathy, diabetic retinopathy, atherosclerosis (e.g., coronary artery disease and peripheral artery disease), diabetic nephropathy, diabetic wound healing, cardiovascular disease, heart failure, ischemia-reperfusion injury, immunological disease, autoimmune disease (e.g., multiple sclerosis, osteoarthritis, and rheumatoid arthritis), sepsis, transplant rejection, cancer (e.g., glioma, breast cancer, liver cancer), pain, liver disease (e.g., hepatitis and liver fibrosis), and lung disease (e.g., acute airway injury and respiratory distress syndrome, chronic obstructive pulmonary disease, emphysema, asthma, cystic fibrosis, and idiopathic pulmonary fibrosis).

In some embodiments, the SMO used in a method described above is as described in the following paragraphs.

The invention also provides splice modulating oligonucleotides (SMOs) including, consisting essentially of, or consisting of 10 to 200 (e.g., 15 to 100, or 20 to 50) nucleotides that are complementary to an exonic or intronic sequence within exon 9, intron 9, or exon 10 of a RAGE pre-mRNA and an optional one or two additional nucleotides. The optional one or two additional nucleotides can be, for example, one or two additional nucleotides added at either or both ends of the SMO, and they can be any nucleotide. For example, they can be any of A, T/U, C, or G, or modified versions or analogs thereof, e.g., as described herein. As described elsewhere herein, the nucleotides of the SMOs can be modified at the base moiety, the sugar moiety, and/or the phosphate backbone.

In some embodiments, the SMO sequence includes or consists of one of SEQ ID NOs: 5 to 2897 or a variant thereof having at least 90% sequence identity to the reference sequence.

In some embodiments, the SMO sequence includes or consists of one of SEQ ID NOs. 5-2897.

In some embodiments, at least one nucleotide in the SMO includes one or more non-naturally occurring modifications including, e.g., at least one of a chemical composition of phosphorothioate 2′-O-methyl, phosphorothioate 2′-MOE, locked nucleic acid (LNA) including thiol-LNA, a constrained moiety, including a constrained ethyl nucleic acid (cEt) or constrained methoxyethyl (cMOE), peptide nucleic acid (PNA), phosphorodiamidate morpholino (PMO), cholesterol, GalNAc, or any combination thereof.

In some embodiments, at least one of the nucleotides of the SMO is a phosphorothioate 2′-O-methyl modified nucleotide.

The invention further provides pharmaceutical compositions including one or more SMO as described above or elsewhere herein.

The invention also provides methods of treating or preventing a disease or condition in a subject that would benefit from altered splicing of RAGE pre-mRNA. The methods include administering to the subject an SMO or composition as described above or elsewhere herein.

In some embodiments, the disease or condition is selected from the group consisting of Alzheimer's disease, amyotrophic lateral sclerosis, diabetes, glucose tolerance, diabetic allodynia and neuropathy, diabetic retinopathy, atherosclerosis (e.g., coronary artery disease and peripheral artery disease), diabetic nephropathy, diabetic wound healing, cardiovascular disease, heart failure, ischemia-reperfusion injury, immunological disease, autoimmune disease (e.g., multiple sclerosis, osteoarthritis, and rheumatoid arthritis), sepsis, transplant rejection, cancer (e.g., glioma, breast cancer, liver cancer), pain, liver disease (e.g., hepatitis and liver fibrosis), and lung disease (e.g., acute airway injury and respiratory distress syndrome, chronic obstructive pulmonary disease, emphysema, asthma, cystic fibrosis, and idiopathic pulmonary fibrosis).

The invention also provides a non-human animal (e.g., a mouse) including a gene encoding human RAGE.

In some embodiments, the gene encoding human RAGE has been introduced into the genome of the non-human animal.

In some embodiments, the gene encoding RAGE of the non-human animal has been edited out, knocked out, and/or replaced with the gene encoding human RAGE.

In some embodiments, the gene encoding human RAGE is a genomic sequence, encoding exons and introns.

In some embodiments, the gene encoding human RAGE is under control of the endogenous RAGE promoter of the non-human animal.

In some embodiments, the non-human animal includes a disease-related mutation. For example, the disease-related mutation may be is in a gene encoding presenilin, SOD1, or the cystic fibrosis membrane transporter (CFTR).

In some embodiments, the non-human animal is an inducible disease model. For example, the non-human animal may be an inducible disease model of a disease selected from the group consisting of Alzheimer's disease, amyotrophic lateral sclerosis, diabetes, glucose tolerance, diabetic allodynia and neuropathy, diabetic retinopathy, atherosclerosis, diabetic nephropathy, diabetic wound healing, cardiovascular disease, heart failure, ischemia-reperfusion injury, immunological disease, autoimmune disease, sepsis, transplant rejection, cancer, pain, liver disease, and lung disease, and optionally effects on physiology or disease are assessed.

The invention further provides methods for identifying or characterizing an SMO directed against human RAGE pre-mRNA, the methods including introducing an SMO into a non-human animal, e.g., as described above and elsewhere herein, and assessing the effects of the SMO on the non-human animal.

In some embodiments, effects on splicing of RAGE pre-mRNA are assessed.

In some embodiments, the non-human animal is a disease model and a feature of the disease is assessed.

In some embodiments, the SMO includes a sequence selected from SEQ ID NOs: 5-2897.

The invention also includes use of an SMO or composition as described herein for carrying out any of the methods described herein.

Other features and advantages of the invention will be apparent from the following detailed description, the drawings, and the claims.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1. Nomenclature and splicing patterns of RAGE isoforms found in human lung or aortic smooth muscle cells. The two most highly expressed isoforms are flRAGE (membrane-bound) and RAGEv1 (soluble). Splice isoforms v2, 3, 7-9, 11, 12, 14, 15, and 17 are potential targets of nonsense mediated decay, however, isoforms such as N-truncated RAGE (RAGEv3) are reported to make protein. Splicing patterns and correlative nomenclature as described previously (Hudson et al., FASEB J. 22(5):1572-1580, 2008).

FIG. 2. Conservation of sequence homology between major species: human, rat, and mouse. Alignment for the 27 nucleotide exon 9, 128 nucleotide intron 9, and 127 nucleotide exon 10 of human, with the 27 nucleotide exon 9, 117 nucleotide intron 9, and 127 nucleotide exon 10 of both rat and mouse sequences. The common flRAGE 5′ exon 9 and 3′ exon 10 splice sites are shown. Differences between human and rodent in alternate 5′ splice site location corresponding to generation of RAGEv1 (and other soluble RAGE isoforms) are also depicted.

FIG. 3. Splicing patterns of SMO-directed RAGE isoforms. A. Detailed splicing pattern of flRAGE (solid lines) and RAGEv1 (dashed lines). B. Other possible splicing patterns generated by SMO-mediated splicing which may or may not correspond to currently known natural splice variants. SMO generated transcripts may include RAGEv1, RAGEv6, RAGEv8, RAGEv9 RAGEv10, RAGEv13, RAGEv15, RAGEv18, RAGEv19. Additionally, read-through into intron 9 could still allow for inclusion of exon 10 (dot-dashed lines), but still produce a truncated soluble RAGE isoform or blocking of exon 10 inclusion with normal splicing at the exon 9 5′ splice site (dotted lines) could cause out of frame truncation of the RAGE protein resulting in either a soluble protein or a transcript that will be funneled to NMD.

FIGS. 4A-4L. RAGE Splicing SMOs. FIG. 4A. Human RAGE target sequences for increasing read-through into intron 9 and/or skipping of exon 10: Exon 9+Intron 9+Exon 10. FIG. 4B. RAGE 25 mer SMO sequences. FIG. 4C. RAGE 24 mer SMO sequences. FIG. 4D. RAGE 23 mer SMO sequences. FIG. 4E. RAGE 22 mer SMO sequences. FIG. 4F. RAGE 21 mer SMO sequences. FIG. 4G. RAGE 20 mer SMO sequences. FIG. 4H. RAGE 19 mer SMO sequences. FIG. 4I. RAGE 18 mer SMO sequences. FIG. 4J. RAGE 17 mer SMO sequences. FIG. 4K. RAGE 16 mer SMO sequences. FIG. 4L. RAGE 15 mer SMO sequences.

### DETAILED DESCRIPTION

The invention provides Splice Modifying Oligonucleotides (SMOs) that can be used to modulate the splicing of pre-mRNA encoding the Receptor for Advanced Glycation End products (RAGE). The SMOs of the invention can, for example, direct RAGE pre-mRNA splicing to (i) increase the generation of mRNA encoding soluble RAGE (sRAGE), (ii) decrease the generation of mRNA encoding full-length RAGE (flRAGE), or (iii) both increase the generation of mRNA encoding sRAGE and decrease the generation of mRNA encoding flRAGE. As noted above, over-activation of RAGE is associated with a number of different diseases and conditions. In addition, sRAGE can beneficially act as a decoy for RAGE ligands, and thus decrease signal transduction through RAGE. Accordingly, the SMOs of the invention can be used in methods for the treatment and prevention of diseases and conditions characterized by, e.g., over-activation of RAGE. The SMOs, compositions, and methods of the invention are described further, below, after a brief description of splicing of RAGE pre-mRNA.

The term “sRAGE” is used throughout to denote all soluble isoforms of the RAGE receptor as a group. The term “syn-sRAGE” denotes a synthetic soluble protein that may be of identical amino acid composition to any soluble RAGE isoforms, particularly RAGEv1. The term “mbRAGE” denotes membrane-bound RAGE, while “flRAGE” denotes full-length RAGE. The term “RAGE” is used in the most general way, where the type of RAGE (e.g., membrane-bound or soluble) can be inferred from the context in which it is used.

Splicing of RAGE pre-mRNA

As a basis for understanding how to modulate splicing of RAGE pre-mRNA, a description of certain aspects of alternative splicing of RAGE pre-mRNA follows.

The most abundant RAGE transcript is the full-length mRNA isoform (“flRAGE”) (NM_001136.4; NP_01127.1), and contains 11 exons, a 5′ untranslated region (UTR), and a short 3′ UTR. The flRAGE transcript is translated into a protein of 404 amino acids (aa), which includes (i) an extracellular region (aa 1-342) comprised of a signal peptide (aa 1-22) and three immunoglobulin (Ig)-like domains, including a V-type domain and partial ligand binding site (aa 23-116) and two C2-type 1/2 domains (aa 124-221 and 227-317); (ii) a single transmembrane domain (aa 343-363); and (iii) a short cytoplasmic tail (aa 364-404) (Neeper et al., J. Biol. Chem. 267(21):14998-15004, 1992). flRAGE is the only RAGE isoform capable of binding all RAGE ligands. The second most prevalent RAGE isoform is a naturally alternatively spliced variant RAGEv1 (also called esRAGE or sRAGE; NM_001206940.1; NP_001193869.1).

The mRNAs of flRAGE and RAGEv1 are identical from exon 1 to the end of exon 9. However, RAGEv1 uses an alternate splice site at the exon 9/intron 9 boundary, which facilitates alternative inclusion of the first 82 nucleotides (nt) of intron 9, skipping of exon 10, and inclusion of exon 11, which contains a polyadenylation sequence (Yonekura et al., Biochem. J. 370(Pt 3):1097-1109, 2003). An “in frame” UGA stop codon at positions 51-53 of intron 9 terminates the coding sequence of RAGEv1. By the alternative inclusion of part of intron 9 in the coding sequence and the premature stop codon, the RAGEv1 protein sequence diverges from flRAGE at amino acid 332, followed by 15 unique amino acids, yielding a truncated protein isoform of 347 amino acids that lacks both the transmembrane domain and cytosolic tail of the 404 amino acid flRAGE (Chuah et al., Int. J. Inflam. 403-460, 2013). Thus, the RAGEv1 isoform can act as a soluble decoy receptor, binding to and clearing ligands from the circulation without activating cell signaling pathways normally associated with ligand binding (Ohe et al., J. Biochem. 147(5):651-659, 2010).

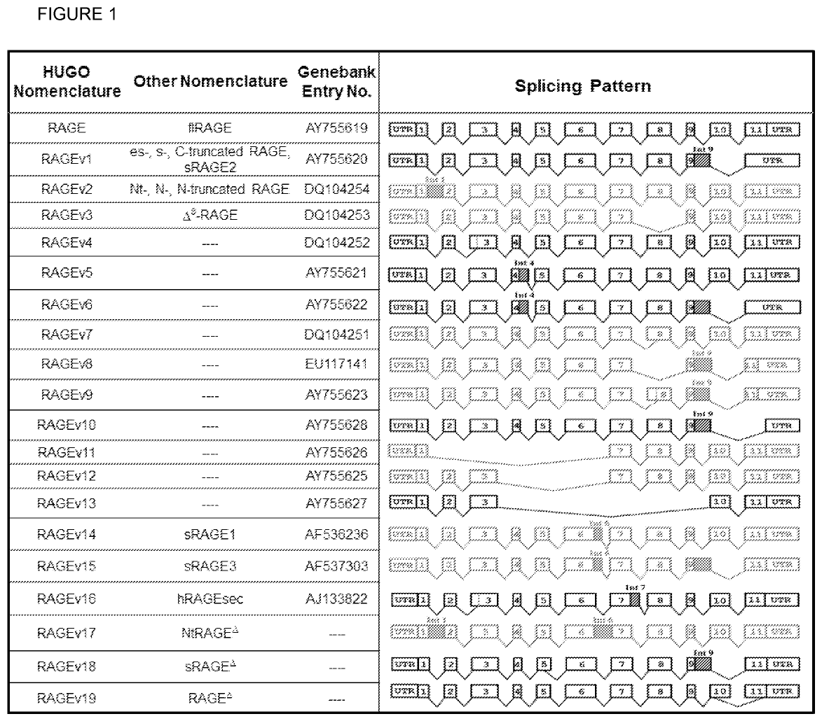

Additional splice isoforms of both membrane-bound and soluble RAGE (generally sRAGE) have been detected (FIG. 1), but all at much lower levels than flRAGE and RAGEv1. For example, RAGEv10 (NM_001206966.1; NP_001193895.1) is a soluble isoform with the same mRNA and resultant protein as RAGEv1, differing only by a shorter 3′ UTR. Also, there are several C-truncated soluble forms of RAGE that arise by post-translational proteolytic cleavage of flRAGE by MMP-9 or ADAM10 metalloproteinase (Chuah et al., Int. J. Inflam. 403-460, 2013; Mahajan et al., Int. J. Cardiol. 2013; Zhang et al., J. Biol. Chem. 283(51):35507-35516, 2008). As is the case for RAGEv1 and RAGEv10, all of the cleaved soluble RAGE isoforms are thought to facilitate the clearance or detoxification of a wide array of ligands associated with human diseases.

### Splice Modulating Oligonucleotides (SMOs)

SMOs are a type antisense oligonucleotide which, when engineered with a particular sequence of the proper chemistry, will bind to a complementary sequence within transcribed pre-mRNA of a target gene and sterically block or weaken interactions between elements of the spliceosome and the pre-mRNA. This results in modulation of the resultant mRNA sequences at a quantitative and/or qualitative level.

Accordingly, an SMO of the invention may be defined generally as a nucleotide sequence (or oligonucleotide), a portion of which is capable of hybridizing with a target nucleic acid to exact an antisense activity on the target nucleic acid. Alternatively, an SMO of the invention can be defined functionally as a nucleotide sequence (or oligonucleotide), at least a portion of which is complementary to and capable of hybridizing with a target nucleic acid sequence (e.g., a RAGE pre-mRNA) to exact a splice modulation in the target RNA of at least, e.g., 5%, 10%, 20%, 30%, 40%, 50%, 75%, 90%, or 100% for a given subject as measured by target RNA levels.

With respect to the SMOs of the invention, the term “splice modulation” refers to molecular manipulation of pre-mRNA splicing to direct a change in the final composition of the mRNA transcript. It is appreciated that complementarity to the target pre-mRNA alone may not be sufficient to produce a functional SMO. The location of SMO binding (e.g., a splicing motif in the pre-mRNA) and thermodynamics of binding at that site, as well as secondary structure of the pre-mRNA or SMO, are among the factors that determine whether splice modulation occurs and the magnitude thereof.

Sequences that can be targeted by SMOs can be selected by those of skill in the art and include, for example, a complementary sequence on a pre-mRNA at an exon or intron splice suppressor or splice enhancer site, at an intron-exon splice site (5′ or 3′), or at a variety of sites on the pre-mRNA containing various other motifs that affect splicing. For example, when an SMO specifically binds to a splice enhancer site, or an intron-exon splice site, the adjacent exon may be excluded from the resulting mRNA. Additionally, an SMO may specifically bind to a splice suppressor site or an intron-exon site, and the adjacent exon may be included in the resulting mRNA. An SMO may further specifically bind to a splice enhancer site or an intron-exon splice site and shift the reading frame of the pre-mRNA so that the resulting protein is truncated. In some cases, the resulting protein is a limited-function or non-functional protein. The location of an exonic or intronic splice enhancer or suppressor motif may be found anywhere within the exon and the flanking introns. Similarly, an SMO may either fully or partially overlap an exonic or intronic splice enhancer or suppressor site in proximity to an intron-exon boundary and/or be complementary to the 3′ or 5′ splice sites.

The sequences of the SMOs of the invention can be described in terms of their relationship to the target pre-mRNA sequences to which they hybridize, and thus to which they are complementary. In a related manner, they can also be described with respect to variant SMO sequences with which they have a given level of sequence identity.

A target RNA (e.g., pre-mRNA, such as RAGE pre-mRNA) splice-modifying interaction guided by oligonucleotides of the invention is highly sequence specific. In general, oligonucleotides having 100% complementarity to a portion of the target pre-mRNA are exposed to target pre-mRNA for blocking of sequence elements within the target pre-mRNA. However, it is appreciated that 100% sequence complementarity between the oligonucleotide and the target pre-mRNA is not required to practice the present invention. Thus, sequence variations that might be expected due to genetic mutation, wobble base pairing, strain polymorphism, or evolutionary divergence may be tolerated. In wobble base pairing, non-Watson-Crick nucleotide pairing occurs in which U can pair with both A and G, so A can be substituted with G, and inosine (I) can pair with any base. For example, oligonucleotide sequences with insertions, deletions, and single point mutations relative to the target sequence may also be effective for SMO-mediated effect on pre-mRNA splicing. Alternatively, oligonucleotide sequences with nucleotide analog substitutions or insertions can be effective for splice modulation. Greater than 60% sequence identity (or complementarity), e.g., greater than 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, and any and all whole or partial increments there between the oligonucleotide and the target RNA, e.g., target pre-mRNA, may be preferred.

Incorporation of nucleotide affinity modifications can allow for a greater number of mismatches compared to an unmodified compound. Certain SMO sequences may be more tolerant to mismatches than other oligonucleotide sequences. Those of ordinary skill in the art can determine an appropriate number of mismatches between oligonucleotides, between an SMO and a target nucleic acid, such as by determining melting temperature (Tm) and evaluating the effect of chemical modifications on the Tm and hybridization stringency (Freier et al., Nucleic Acids Research 25, 22:4429-4443, 1997).

With respect to an SMO of the invention, the term “hybridize” or “hybridizing” refers to the association between two single-stranded nucleotide molecules of sufficiently complementary sequences to permit such hybridization under pre-determined conditions generally used in the art (including, e.g., physiological conditions). In particular, the term refers to hybridization of an SMO with a substantially complementary sequence contained within a complementary sequence of a target complementary sequence of the RAGE pre-mRNA molecule, to the substantial exclusion of hybridization of the SMO with a pre-mRNA that has a non-complementary sequence. Appropriate conditions enabling specific hybridization of single stranded nucleic acid molecules of varying complementarity are well known in the art. It is appreciated that these conditions are largely dictated by cellular conditions for in vivo applications.

With respect to the SMOs of the invention, the term “complementary” or “complementarity” refers to a degree of antiparallel relationship between a strand of SMO and a pre-mRNA molecule. In some instances, the complementarity between an SMO of the invention and a pre-mRNA is between 60% and 100%, e.g., at least 60%, 65%, 70%, 75%, 80%, 85%, 90%, 91%, 92%, 93%, 94%, 95%, 96%, 97%, 98%, 99%, or 100%.

Further, with respect to SMO variants of the invention, the term “sequence identity” or “identity” in the context of two nucleic acid sequences (e.g., an SMO and a variant thereof) makes reference to a specified percentage of residues in the two sequences that are the same when aligned by sequence comparison algorithms or by visual inspection. For example, sequence identity may be used to reference a specified percentage of residues that are the same across the entirety of the two sequences when aligned. In certain embodiments, the term “substantial identity” of polynucleotide sequences means that a polynucleotide includes a sequence that has at least 60%, 65%, 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters.

Sequence identity, including determination of sequence complementarity or homology for nucleic acid sequences, can be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=number of identical positions/total number of positions×100), optionally penalizing the score for the number of gaps introduced and/or length of gaps introduced. The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A non-limiting example of a local alignment algorithm utilized for the comparison of sequences is the algorithm of Karlin and Altschul, Proc. Natl. Acad. Sci. U.S.A. 87:2264-68, 19990, modified as in Karlin and Altschul, Proc. Natl. Acad. Sci. U.S.A. 90:5873-77, 1993. Such an algorithm is incorporated into the BLAST programs (version 2.0) of Altschul et al., J. Mol. Biol. 215:403-410, 1990.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., Nucleic Acids Res. 25(17):3389-3402, 1997. In another embodiment, the alignment is optimized by introducing appropriate gaps and percent identity is determined over the entire length of the sequences aligned (i.e., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

In another embodiment, the sequence identity for two sequences is based on the greatest number of consecutive identical nucleotides between the two sequences (without inserting gaps). For example, the percent sequence identity between Sequence A and B below would be 87.5% (Sequence B is 14/16 identical to Sequence A), whereas the percent sequence identity between Sequence A and C would be 37.25% (Sequence C is 6/16 identical to Sequence A).

Example Sequence A:
(SEQ ID NO: 2898)
GCATGCATGCATGCAT
Example Sequence B:
(SEQ ID NO: 2899)
GCATGCATGCATGC
Example Sequence C:
(SEQ ID NO: 2900)
GCATTTGCAGCAGC

As used herein, a sequence is identical to an SMO sequence disclosed herein if it has the same nucleobase pairing ability. This identity may be over the entire length of the nucleotide sequence, or in a portion of the nucleotide sequence, e.g., nucleobases 1-20 of a 300-mer may be compared to a 20-mer to determine percent identity of the nucleic acid to the SEQ ID NO described herein. Percent identity is calculated according to the number of nucleotide bases that have identical base pairing corresponding to the SEQ ID NO or SMO compound to which it is being compared. The non-identical bases may be adjacent to each other, dispersed throughout the nucleotide sequence, or both. For example, an 18-mer having the same sequence as nucleobases 3-20 of a 24-mer SMO is 75% identical to the 24-mer SMO. Alternatively, a 24-mer containing six nucleobases not identical to another 24-mer is also 75% identical to the 24-mer. Similarly a 15-mer having the same sequence as nucleobases 1-15 of a 100-mer is 15% identical to the 100-mer. Such calculations are well within the ability of those skilled in the art.

It is further understood by those skilled in the art that a nucleic acid sequence need not have an identical sequence to those described herein to function similarly to the SMO compound described herein. Shortened versions of SMO compounds taught herein, or non-identical versions of the SMO compounds taught herein, are also provided. Non-identical versions can include at least one base replaced with a different base with different pairing activity (e.g., G can be replaced by C, A, or T), wobble base pairing, or sequences are those wherein each base does not have the same pairing activity (e.g., by the nucleic acid sequence being shorter or having at least one abasic site) as the SMOs disclosed herein.

SMOs of the invention are typically about, for example, 10-200 nucleotides long (e.g., 12-175, 14-150, 15-125, 20-100, or 25-75). In specific examples, the SMO sequence is 14 to about 26 nucleotides long (e.g., about 14, 15, 16, 17, 18, 19, 20, 21, 22, 23, 24, 25, or 26 nucleotides long). In some instances, only a portion of an SMO hybridizes to the pre-mRNA, and that portion has the requisite level of complementarity to support hybridization of the SMO to the pre-mRNA. Thus, for example, a portion of an SMO that hybridizes to pre-mRNA may comprise, e.g., about 12-100 nucleotides, while the SMO molecule itself may comprise additional nucleotides, e.g., 1-100, 2-80, 3-70, 4-60, 5-50, 6-40, 7-30, 8-20, or 10-15 additional nucleotides.

Specifically in regard to RAGE pre-mRNA, SMOs targeting RAGE pre-mRNA sequences are complementary to the RAGE pre-mRNA sequences, such that they bind to the target sequences sufficiently to block or otherwise alter a splicing event, as described herein.

In various examples, the invention provides “dual mechanism” SMOs that simultaneously (i) reduce the expression of a membrane-bound RAGE protein (e.g., flRAGE), and (ii) increase the expression of a secreted sRAGE protein (e.g., RAGEv1), which can act as a decoy by binding ligands (e.g., AGEs) of membrane bound active RAGE isoforms, but is not capable of transducing deleterious ligand-RAGE signaling events. In these examples, the SMO-mediated increase in ligand-sRAGE binding can greatly decrease the deleterious RAGE ligands from stimulating pathological RAGE signaling, not only by binding them, but also by ridding them from circulation. The invention also provides “single mechanism” SMOs with properties to reduce the expression of a membrane-bound RAGE protein (e.g., flRAGE), or increase the expression of a secreted sRAGE protein (e.g., RAGEv1), which can act as a decoy by binding ligands (predominantly AGEs) of membrane bound active RAGE isoforms, but is not capable of transducing deleterious ligand-RAGE signaling events.

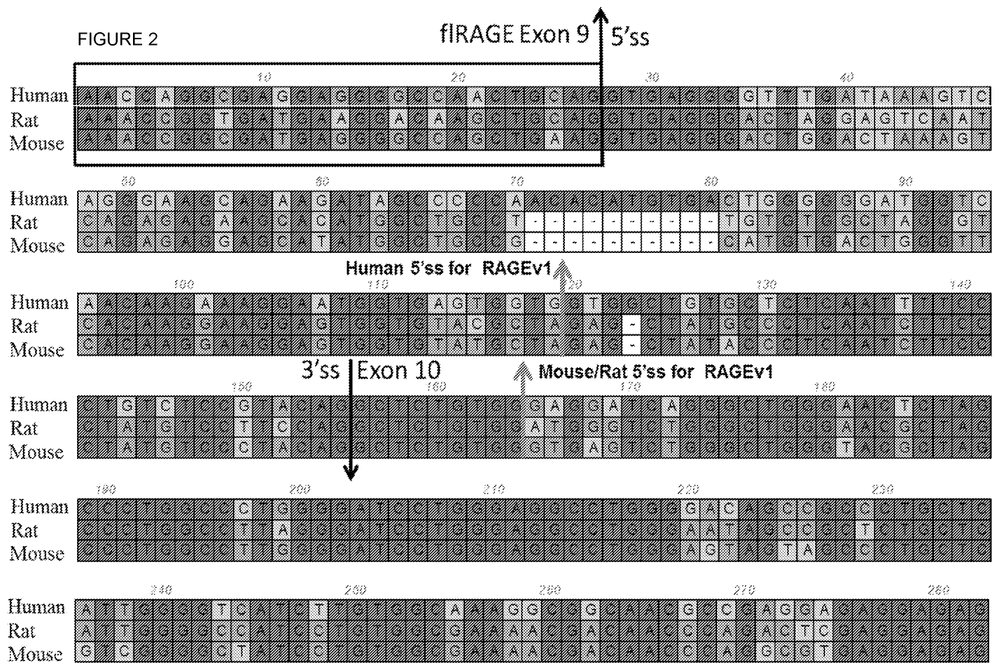

Accordingly, in various examples, certain SMOs of the invention can be used to increase the generation of mRNA encoding sRAGE (e.g., RAGEv1), relative to the amount of flRAGE mRNA produced. As noted above, a single exon 9-intron 9 splicing event can determine whether flRAGE or an sRAGE (i.e., RAGEv1) mRNA is produced. Accordingly, SMOs directed at this particular splicing event can be used, for example, to decrease flRAGE and/or to increase sRAGE (e.g., RAGEv1) expression. By controlling alternative splicing of the RAGE transcript, receptor binding, signaling properties, and ligand clearance, can be regulated rather than simply downregulating or antagonizing the RAGE receptor. SMOs can be designed based on, e.g., the consensus sequence of RAGE (AGER: HGNC #320, OMIM: 600214; Genbank KR711244.1), including upstream and downstream nucleotides (see, e.g., FIG. 2).

In certain embodiments, the SMO comprises a sequence designed to modulate the splicing of exon/intron 9 in the RAGE pre-mRNA. In certain embodiments, the SMO comprises a sequence designed to include a portion of intron 9 in a resulting RAGE mRNA. In certain embodiments, the SMO comprises a sequence designed to exclude exon 10 in a resulting RAGE mRNA. In certain embodiments, the SMO comprises a sequence that specifically binds to a 3′ or 5′ splice site of 9. In certain embodiments, the SMO comprises a sequence that specifically binds to an exon 9 exonic splice enhancer (ESE) sequence. In certain embodiments, the SMO comprises a sequence that specifically binds to an exon 9 intronic splice enhancer (ISE) sequence. In certain embodiments, the SMO comprises a sequence that specifically binds to an exon 9 intronic splice silencer (ISS) sequence. In certain embodiments, the SMO comprises a sequence that specifically binds to an exon 9 exonic splice silencer (ESS) sequence. In certain embodiments, the SMO comprises a sequence that specifically binds to exon 9 of the RAGE pre-mRNA (e.g., binds to a complementary sequence in exon 9 (either partially or wholly within exon9)).

In certain embodiments, the SMO comprises a sequence that has at least about 60% complementarity with a sequence of one of SEQ ID NOs: 1-4. In certain embodiments, the sequence has at least about 60, 65, 70, 75, 80, 81, 82, 83, 84, 85, 86, 87, 88, 89, 90, 91, 92, 93, 94, 95, 96, 97, 98, 99, or 100% complementarity with a sequence of one of SEQ ID NOs: 1-4.

In some embodiments, the SMO sequence is about 10-200 nucleotides long (e.g., 12-175, 14-150, 15-125, 20-100, or 25-75 nucleotides long). For example, the SMO sequence may be about 14, 15, 16, 17, 18, 19, 20, 21, 22, 23, 24, 25 or 26 nucleotides long. In certain embodiments, the SMO is about 15 to about 26 nucleotides long.

In certain embodiments, the SMO comprises or consists of about 14 to about 26 nucleotides, and comprises or consists of between about 6 and 24 contiguous nucleotides (i.e., about 6, 7, 8, 9, 10, 11, 12, 13, 14, 15, 16, 17, 18, 19, 20, 21, 22, 23 or 24 contiguous nucleotides) of any one of SEQ ID NOs: 5-2897. In certain embodiments, the SMO comprises between about 10 to about 24 contiguous nucleotides of any one of SEQ ID NOs: 5-2897. In certain embodiments, the SMO comprises about 11, 12, 13, 14, 15, 16, 17, 18, 19, 20, 21, 22, 23 or 24 contiguous nucleotides of any one of SEQ ID NOs: 5-2897.

In certain embodiments, the SMO comprises a sequence that has at least 60% sequence identity with any one of SEQ ID NOs: 5-2897. In certain embodiments, the sequence has at least 65, 70, 75, 80, 81, 82, 83, 84, 85, 86, 87, 88, 89, 90, 91, 92, 93, 94, 95, 96, 97, 98, 99 or 100% sequence identity with any one of SEQ ID NOs: 5-2897. In certain embodiments, the sequence is selected from any one of SEQ ID NOs: 5-2897.

Certain embodiments of the invention provide a composition comprising an SMO described herein. In certain embodiments, the composition is a pharmaceutical composition. In certain embodiments, the pharmaceutical composition comprises a pharmaceutically acceptable carrier.

### Synthesis of SMOs

Oligonucleotides of the invention (e.g., SMOs) can be synthesized using procedures known in the art including, e.g., chemical synthesis, enzymatic ligation, organic synthesis, and biological synthesis. In one example, an RNA molecule, e.g., an SMO, is prepared chemically (see, e.g., Verma and Eckstein, Ann. Rev. Biochem. 67:99-134, 1998). RNA can optionally be purified from a mixture by extraction with a solvent or resin, precipitation, electrophoresis, chromatography, or a combination thereof. Alternatively, the RNA may be used with no or a minimum of purification to avoid losses due to sample processing.

### Modifications of SMOs

Oligonucleotides of the invention (e.g., SMOs) can be modified to improve stability in serum or growth medium for cell cultures, or otherwise to enhance stability during delivery to subjects and/or cell cultures.

In order to enhance stability, 3′-residues can be stabilized against degradation, e.g., they can be selected such that they consist of purine nucleotides, particularly adenosine or guanosine nucleotides. Alternatively, substitution of pyrimidine nucleotides by modified analogues, e.g., substitution of uridine by 2′-deoxythymidine, or cytosine by 5′-methylcytosine, can be tolerated without affecting the efficiency of oligonucleotide reagent-induced modulation of splice site selection. For example, the absence of a 2′ hydroxyl may significantly enhance the nuclease resistance of the oligonucleotides.

The SMOs can include one or more modified nucleotide analogue, which may optionally be located at a position(s) that does not substantially affect target-specific activity, e.g., the splice site selection modulating activity is not substantially affected, e.g., in a region at the 5′-end and/or the 3′-end of the SMO molecule. In particular examples, the ends are stabilized by the incorporation of one or more modified nucleotide analogue.

Exemplary nucleotide analogues that can be included in the SMOs are sugar- and/or backbone-modified ribonucleotides. For example, the phosphodiester linkages of natural RNA can be modified to include at least one of a nitrogen or sulfur heteroatom. In certain examples of backbone-modified ribonucleotides, the phosphoester group connecting to adjacent ribonucleotides is replaced by a modified group, e.g., of phosphothioate group. Thus, in various examples, in sugar-modified ribonucleotides, the 2′ OH-group is replaced by a group selected from: CH₃, CH₂CH₂OCH₃, H, OR, R, halo, SH, SR, NH₂, NHR, NR₂, or ON, wherein R is C₁-C₆ alkyl, alkenyl or alkynyl and halo is F, Cl, Br, or I.

Other examples include nucleobase-modified ribonucleotides containing at least one non-naturally occurring nucleobase instead of a naturally occurring nucleobase. Bases may be modified, for example, to block the activity of adenosine deaminase. Exemplary modified nucleobases include, for example, phosphorothioate derivatives and acridine substituted nucleotides, 2′-O-methyl substitutions, 2′-O-(2methoxyethyl) substitutions 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluraci I₅ 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine. It should be noted that the above-listed modifications can be combined.

Oligonucleotides of the invention (e.g., SMOs) also can be modified with chemical moieties (e.g., cholesterol) that improve in vivo pharmacological properties of the oligonucleotides.

Oligonucleotides of the invention can be α-anomeric nucleic acid molecules, which form specific double-stranded hybrids with complementary RNA in which, contrary to the usual α-units, the strands run parallel to each other (Gaultier et al., Nucleic Acids Res. 15:6625-6641, 1987). The oligonucleotides can also include 2′-O-methylribonucleotide (Inoue et al., 1987, Nucleic Acids Res. 15:6131-6148) or a chimeric RNA-DNA analogue (Inoue et al., 1987, FEBS Lett. 215:327-330).

In various embodiments, the oligonucleotides of the invention can be modified at the base moiety, sugar moiety, or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., Bioorg. Med. Chem. 4(1): 5-23, 1996). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The synthesis of PNA oligomers can be performed using standard solid phase peptide synthesis protocols as described in Hyrup et al., 1996, supra; Perry-O'Keefe et al., Proc. Natl. Acad. Sci. U.S.A. 93:14670-14675, 1996. In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup, 1996, supra, and Finn et al., Nucleic Acids Res. 24(17):3357-3363, 1996.

The oligonucleotides of the invention can also be formulated as morpholino oligonucleotides. In such embodiments, the riboside moiety of each subunit of an oligonucleotide of the oligonucleotide is converted to a morpholine moiety (morpholine=C₄H9NO; Heasman, Dev. Biol. 243, 209-214, 2002).

A further oligonucleotide modification includes Locked Nucleic Acids (LNAs), in which the 2′-hydroxyl group is linked to the 3′ or 4′ carbon atom of the sugar ring thereby forming a bicyclic sugar moiety. The linkage can be a methylene (˜CH₂˜)ₙ group bridging the 2′ oxygen atom and the 4′ carbon atom wherein n is 1 or 2. LNAs and preparation thereof are described in WO 98/39352 and WO 99/14226. In other embodiments, the oligonucleotide can include other appended groups such as peptides (e.g., for targeting host cell receptors in vivo), or agents facilitating transport across the cell membrane (see, e.g., Letsinger et al., Proc. Natl. Acad. Sci. U.S.A. 86:6553-6556, 1989; Lemaitre et al., Proc. Natl. Acad. Sci. USA 84:648-652, 1987; WO 88/09810) or the blood-brain barrier (see, e.g., WO 89/10134). In addition, oligonucleotides can be modified with hybridization-triggered cleavage agents (see, e.g., Krol et al., Bio/Techniques 6:958-976, 1988) or intercalating agents (see, e.g., Zon, Pharm. Res. 5:539-549, 1988). To this end, the oligonucleotide can be conjugated to another molecule, e.g., a peptide, hybridization triggered cross-linking agent, transport agent, hybridization-triggered cleavage agent, etc.

Within the oligonucleotides (e.g., oligoribonucleotides) of the invention, as few as one or as many as all of the nucleotides of the oligonucleotide can be modified. For example, a 20-mer oligonucleotide (e.g., oligoribonucleotide) of the invention can contain 1, 2, 3, 4, 5, 6, 7, 8, 9, 10, 11, 12, 13, 14, 15, 16, 17, 18, 19, or 20 modified nucleotides. In various examples, the modified oligonucleotides (e.g., oligoribonucleotides) contain as few modified nucleotides as are necessary to achieve a desired level of in vivo stability and/or bio-accessibility while maintaining cost effectiveness. SMOs of the invention include oligonucleotides synthesized to include any combination of modified bases disclosed herein in order to optimize function. In one example, an SMO of the invention includes at least two different modified bases. In another example, an SMO of the invention includes alternating 2′-O-methyl substitutions and bicyclic sugar moieties (e.g. LNA bases).

In certain embodiments, the SMO comprises at least one nucleotide that contains a non-naturally occurring modification comprising at least one of a chemical composition of phosphorothioate 2′-O-methyl (2′OMe), phosphorothioate 2′-methoxyethyl (2′-O-MOE), locked nucleic acid (LNA) peptide nucleic acid (PNA), phosphorodiamindate morpholino (PMO), or any combination thereof.

In certain embodiments, the SMO comprises at least one 2′-O-methyl nucleotide. In certain embodiments, the SMO comprises at least two 2′-O-methyl nucleotides. In certain embodiments, the SMO comprises at least three 2′-O-methyl nucleotides. In certain embodiments, at least about 10, 20, 30, 40, 50, 60, 70, 80, 90 or 100% of the SMO nucleotides are 2′-O-methyl modified.

In certain embodiments, the SMO comprises at least one nucleotide with a phosphorothioate linkage. In certain embodiments, the SMO comprises at least two nucleotides with phosphorothioate linkages. In certain embodiments, the SMO comprises at least three nucleotides with phosphorothioate linkages. In certain embodiments, at least about 10, 20, 30, 40, 50, 60, 70, 80, 90 or 100% of the SMO nucleotides comprise phosphorothioate linkages.

In certain embodiments, the SMO comprises at least one phosphorothioate 2′-O-methyl modified nucleotide. In certain embodiments, the SMO comprises at least two phosphorothioate 2′-O-methyl modified nucleotides. In certain embodiments, the SMO comprises at least three phosphorothioate 2′-O-methyl modified nucleotides. In certain embodiments, at least about 10, 20, 30, 40, 50, 60, 70, 80, 90 or 100% of the SMO nucleotides are phosphorothioate 2′-O-methyl modified.

In certain embodiments, modifications include a bicyclic sugar moiety similar to LNAs (see U.S. Pat. No. 6,043,060) where the bridge is a single methylene group which connect the 3′-hydroxyl group to the 4′ carbon atom of the sugar ring thereby forming a 3′-C,4′-C-oxymethylene linkage. In certain embodiments oligonucleotide modifications include cyclohexene nucleic acids (CeNA), in which the furanose ring of a DNA or RNA molecule is replaced with a cyclohexenyl ring to increase stability of the resulting complexes with RNA and DNA complements (Wang et al., Nucleic Acids 20(4-7):785-788, 2001). In certain embodiments oligonucleotide modifications include constrained 2′O-methoxyethyl (cMOE) in which the ethyl group of 2′O-methoxyethyl is connected to the 4′ position of the furanose ring and constrained ethyl (cEt), in which the ethyl group of the cMOE is replaced with a methyl group that is similarly connected to the 4′ position of the furanose ring (Seth et al., Nucleic Acids Symp. Ser. (Oxf)(52):553-554, 2008). In certain embodiments other bicyclic and tricyclic nucleoside analogs are included in the SMO.

### Methods of Use

Methods of Modulating RAGE pre-mRNA Splicing

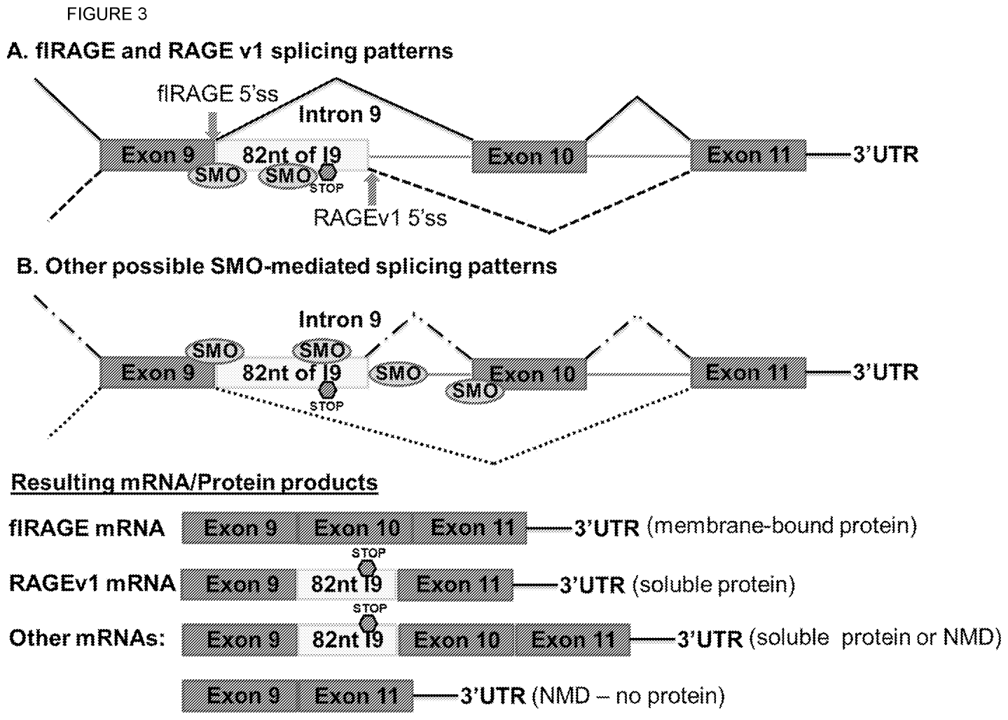

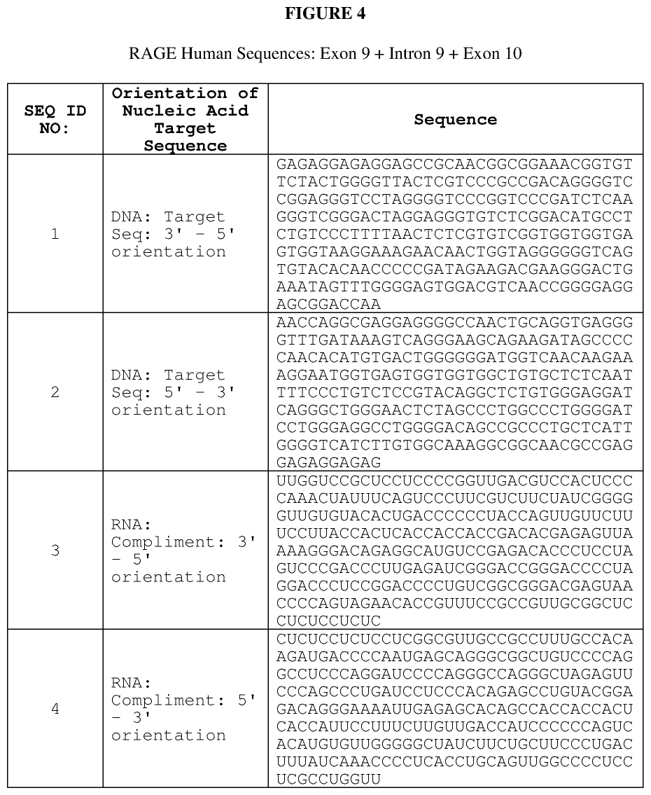

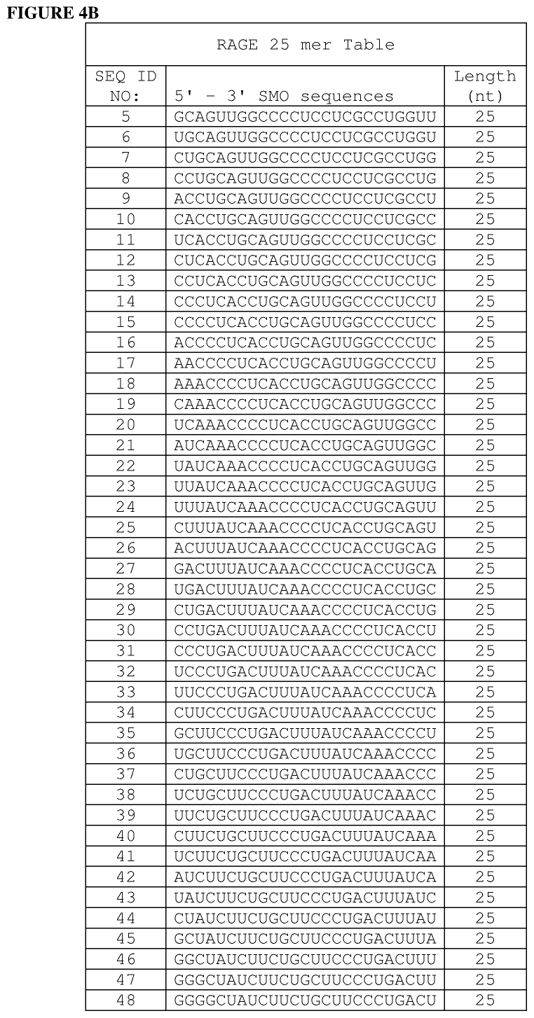

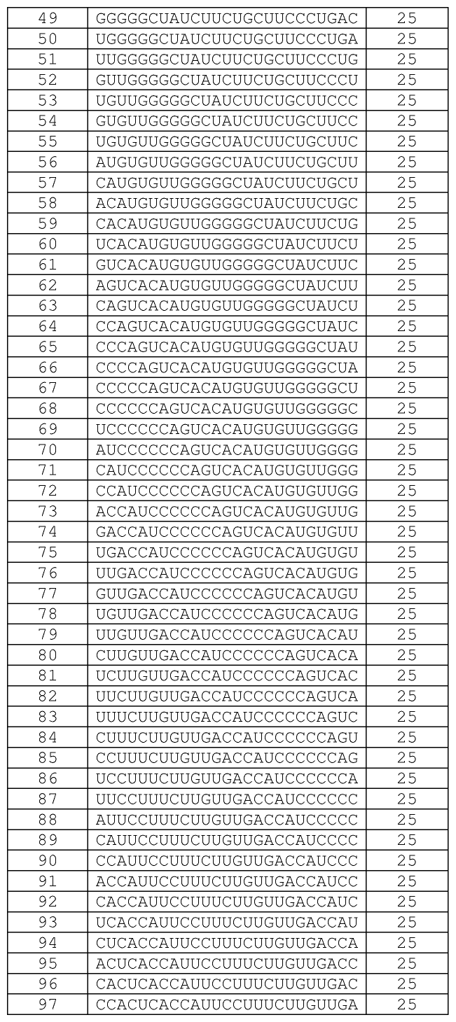

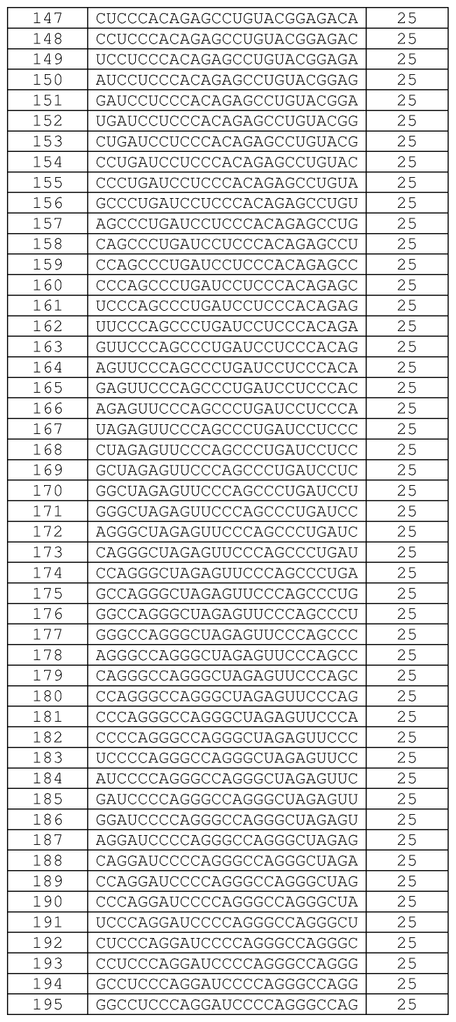

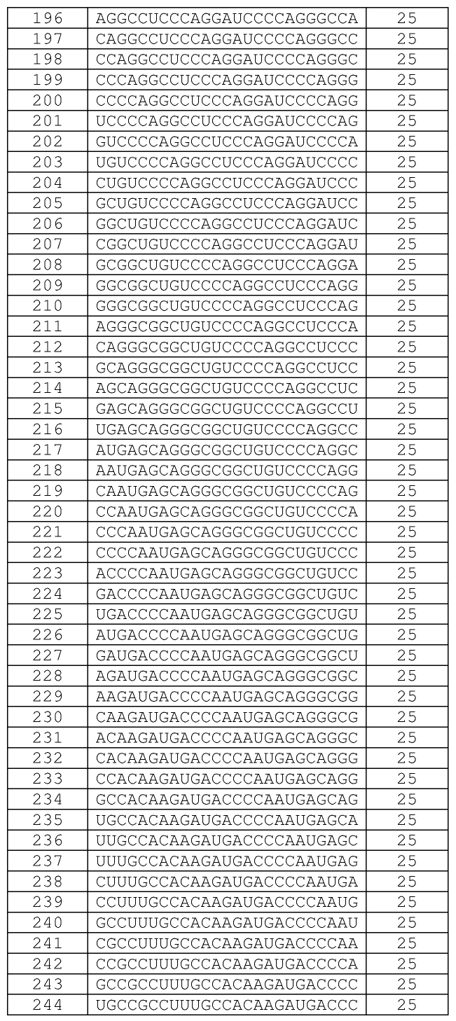

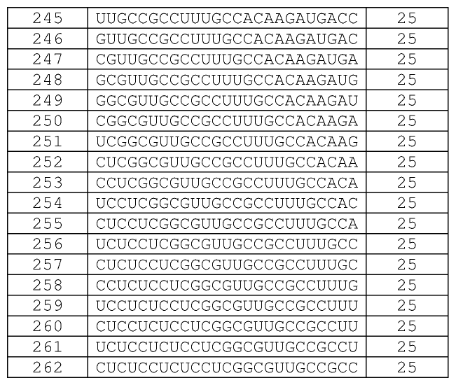

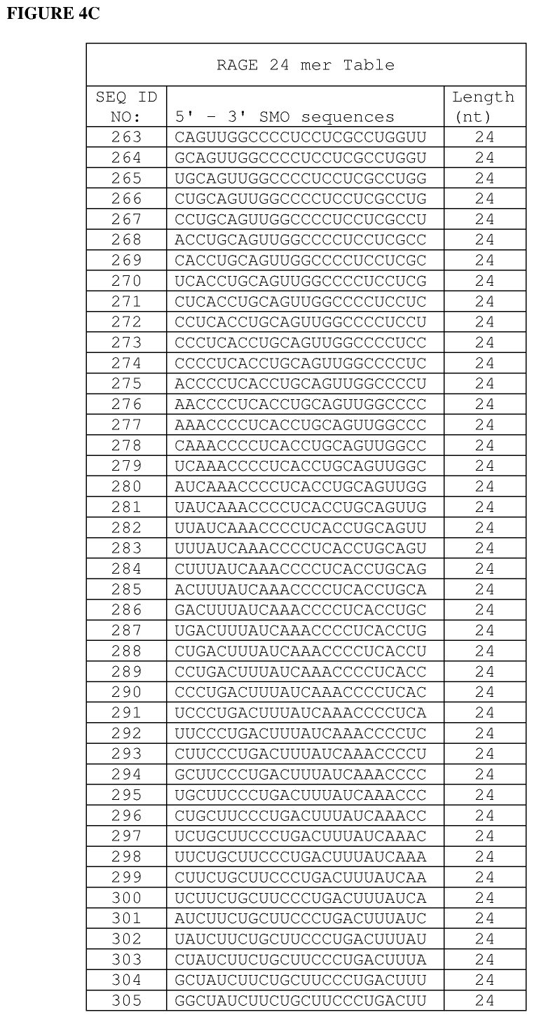

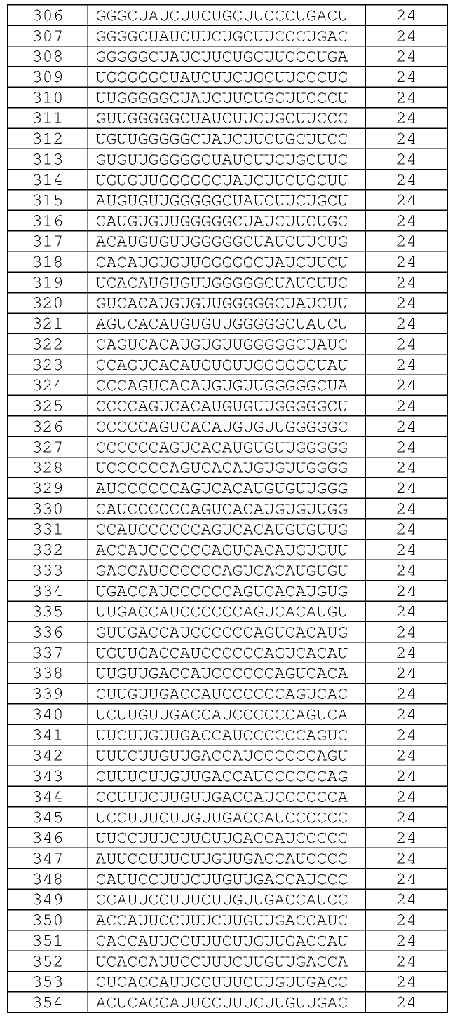

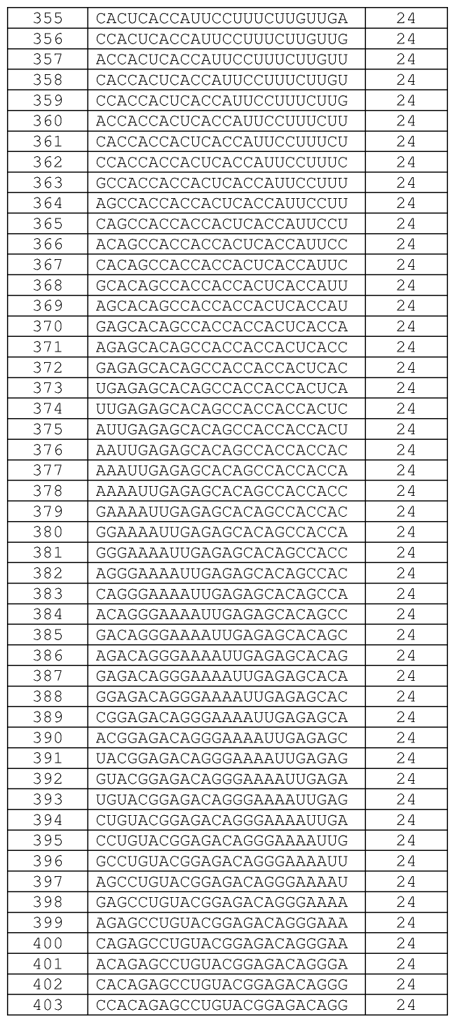

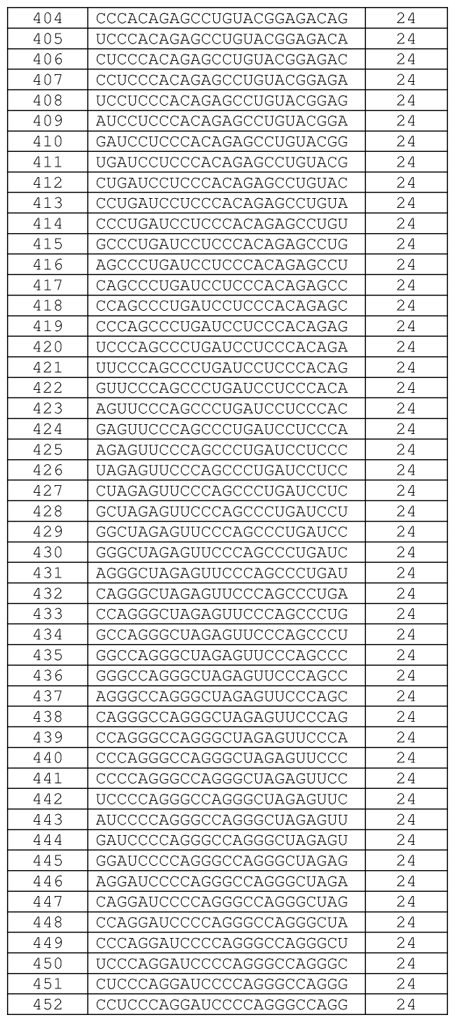

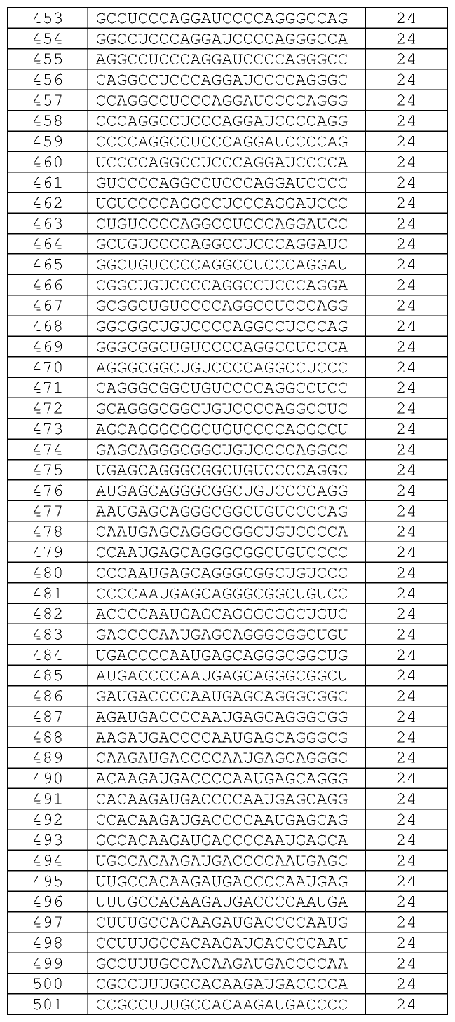

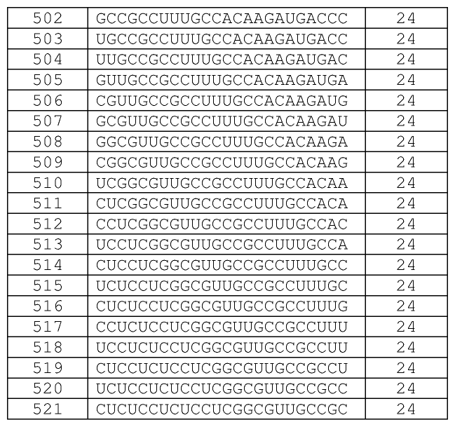

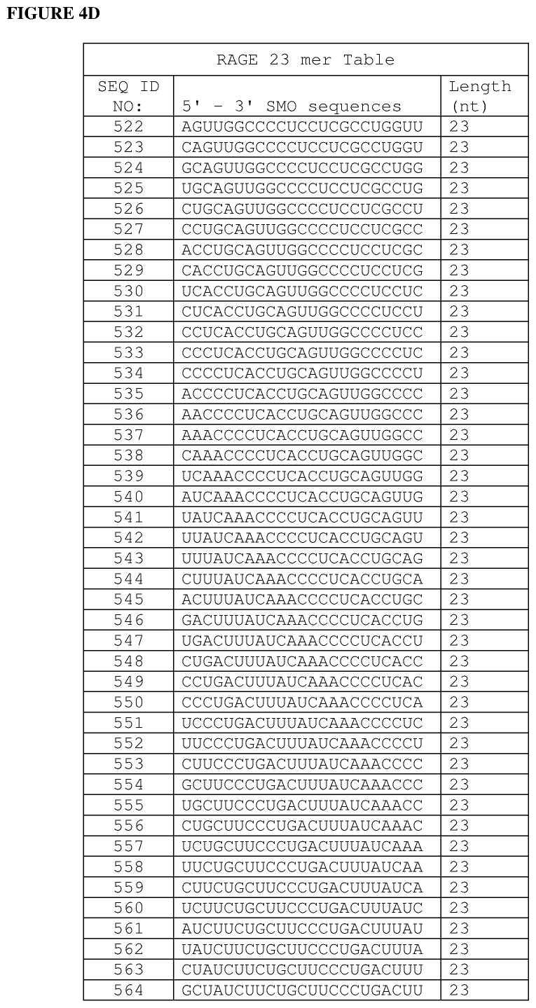

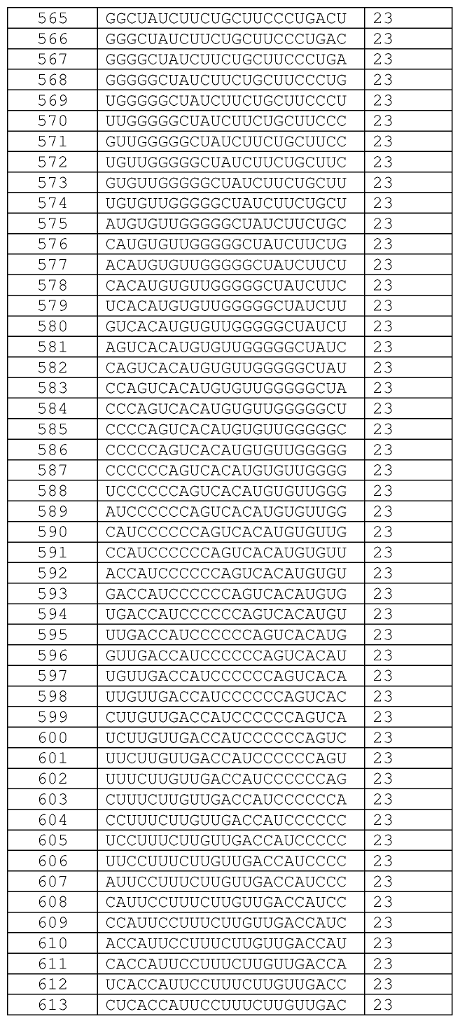

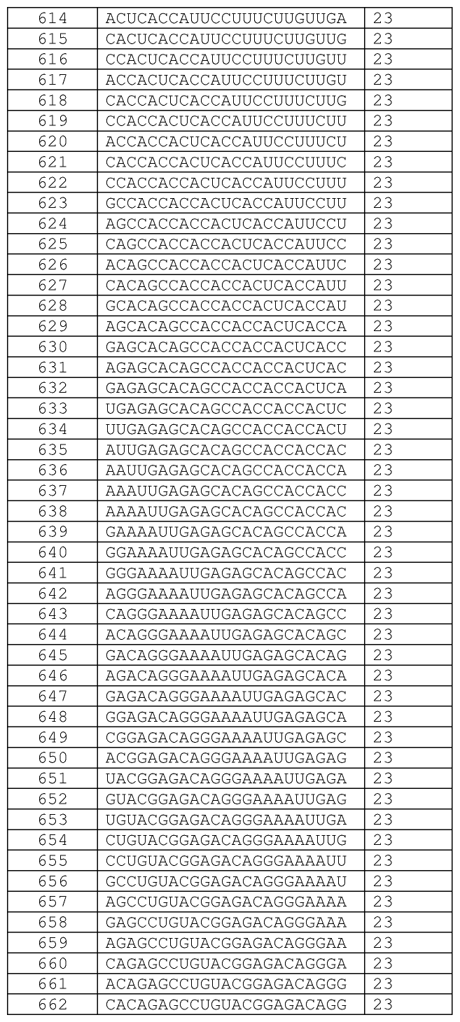

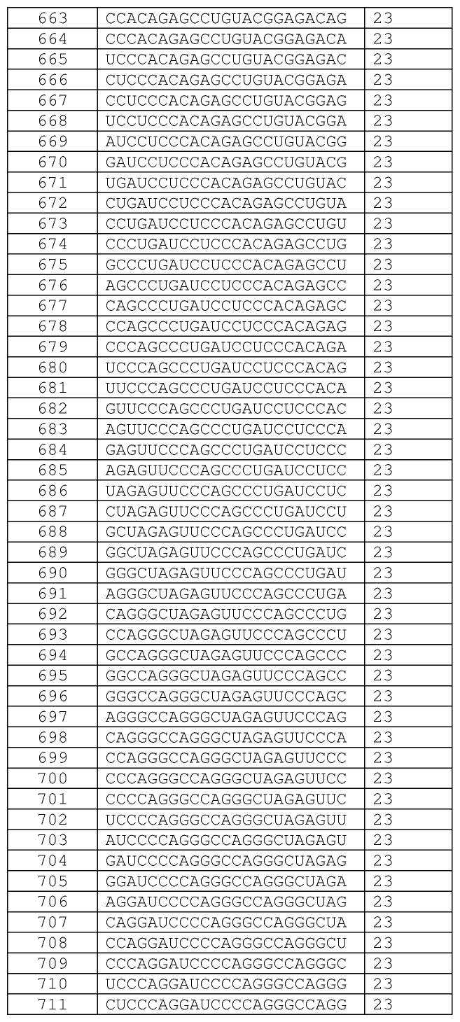

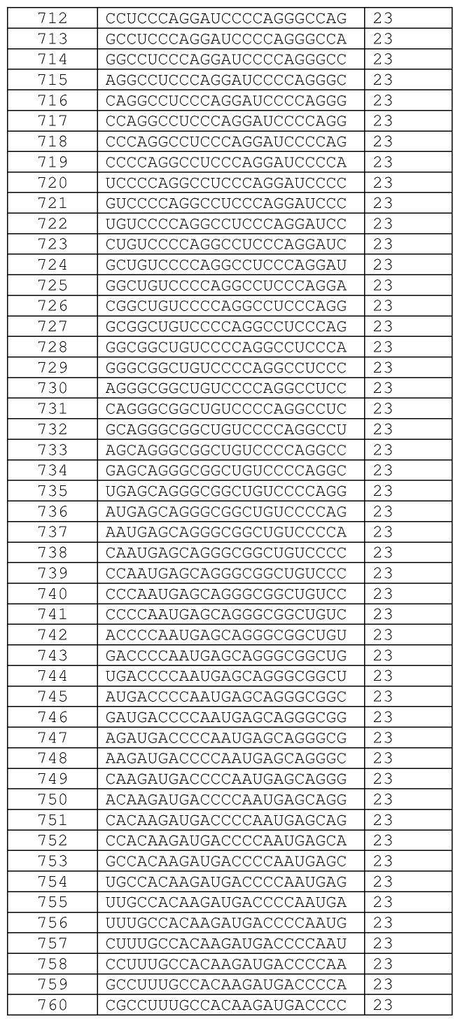

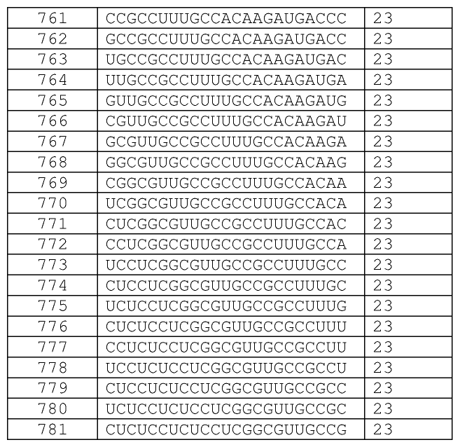

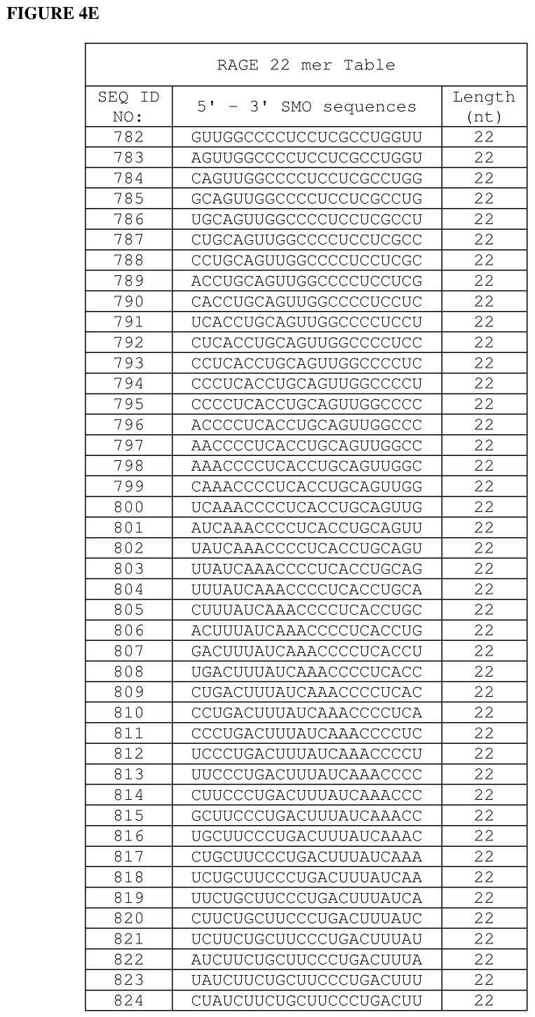

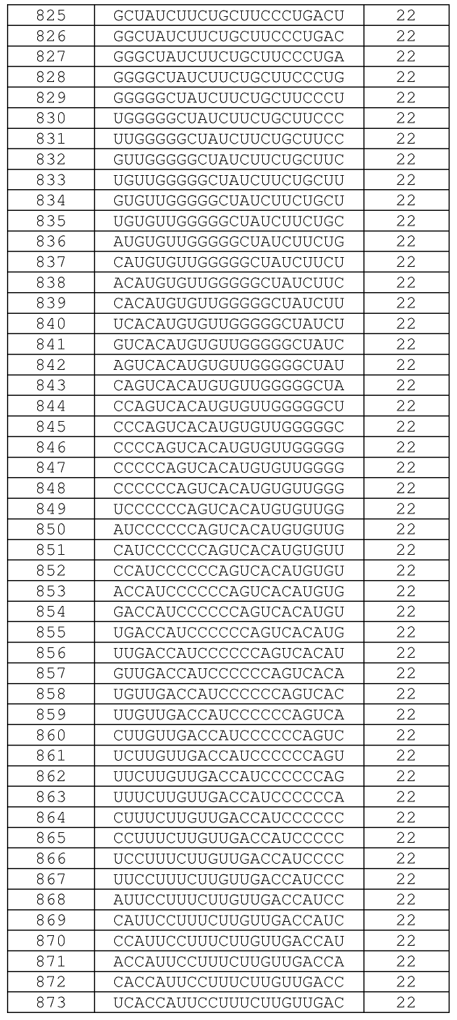

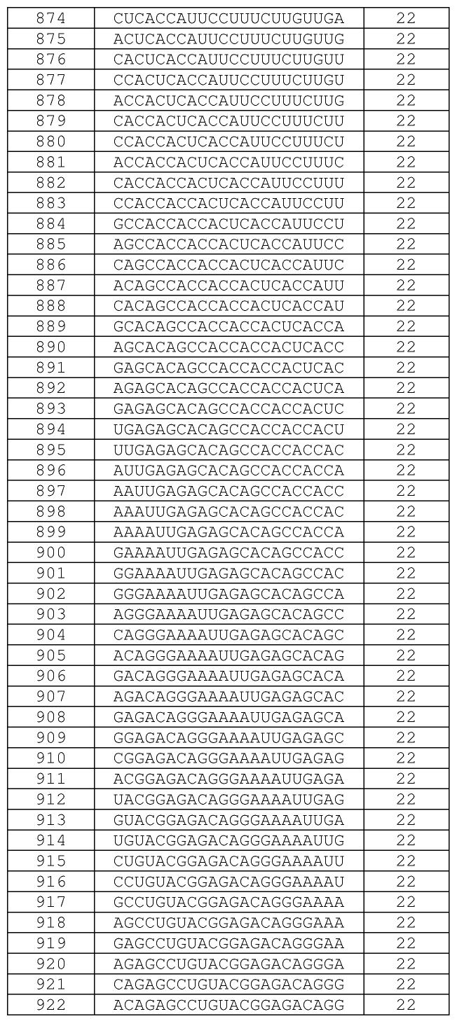

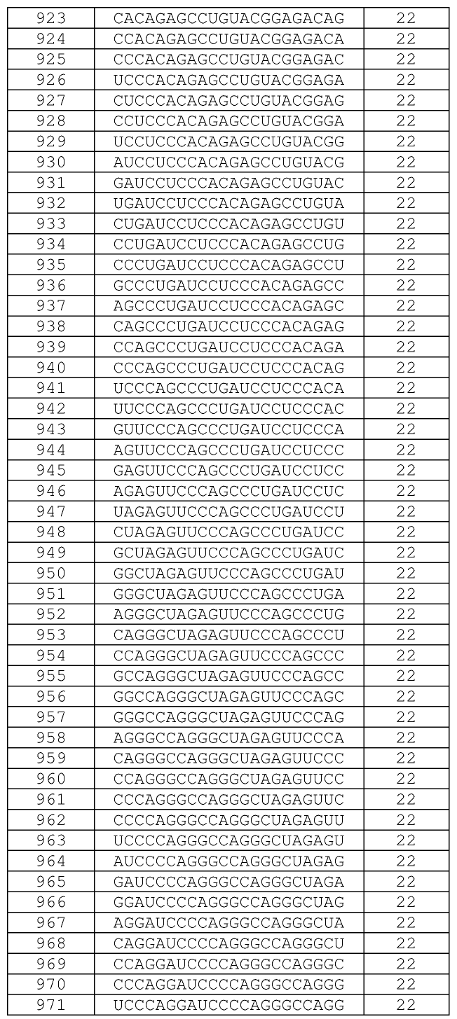

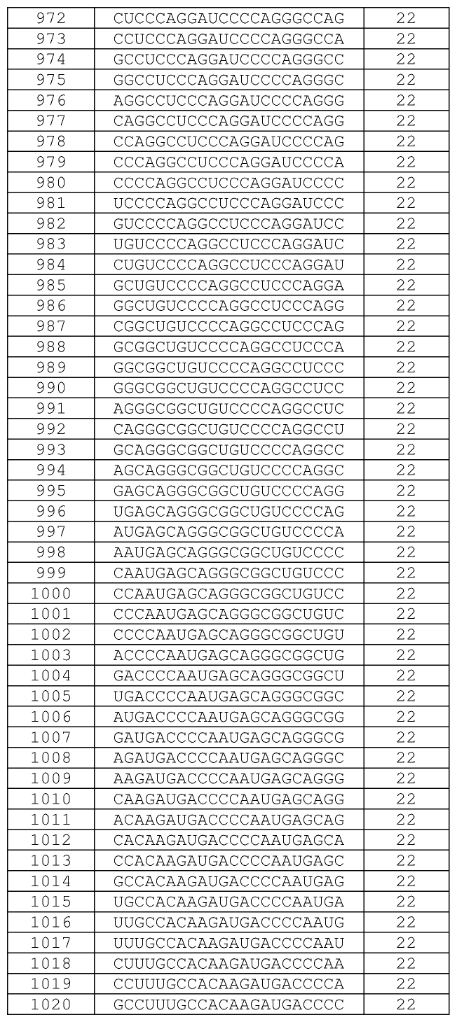

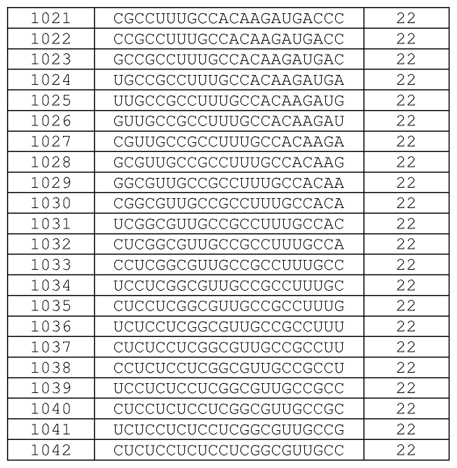

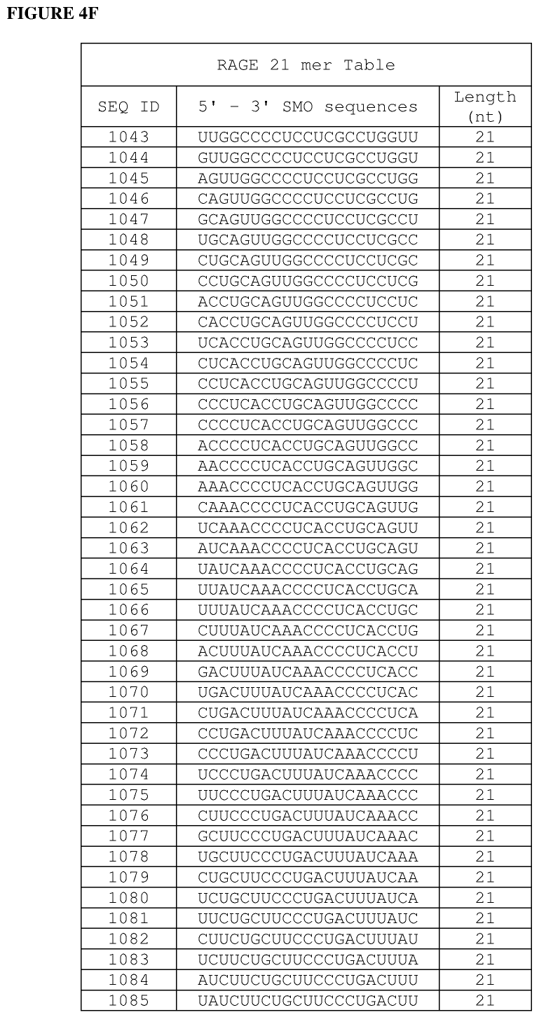

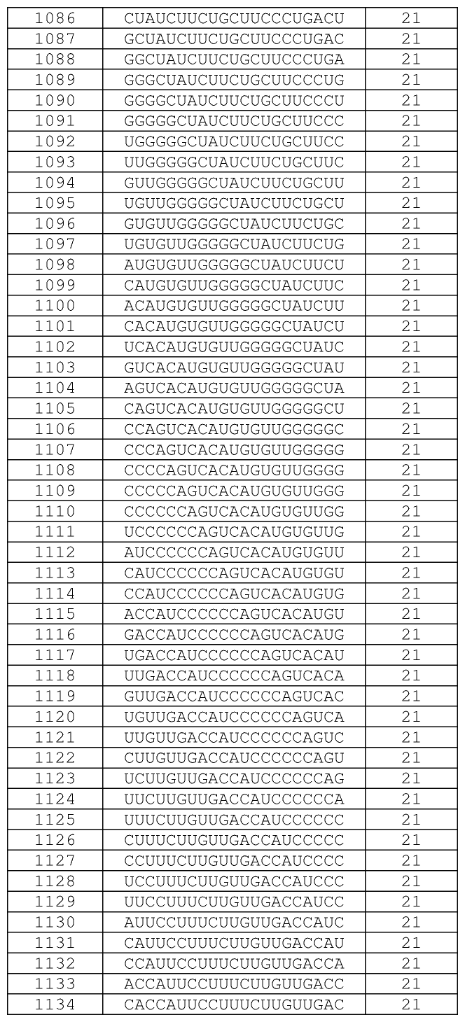

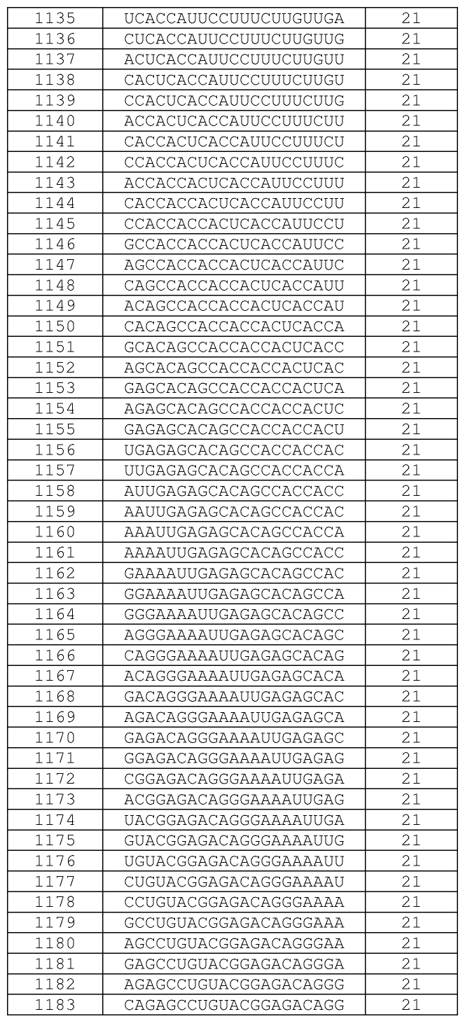

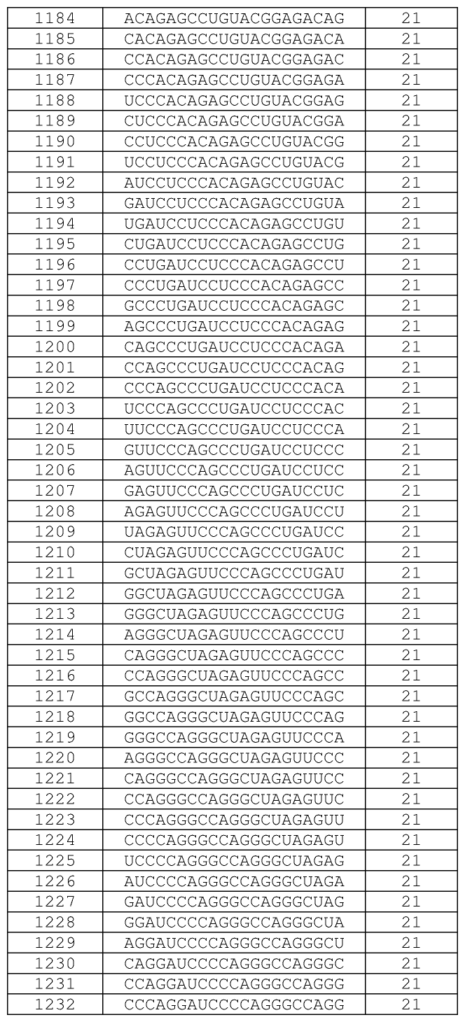

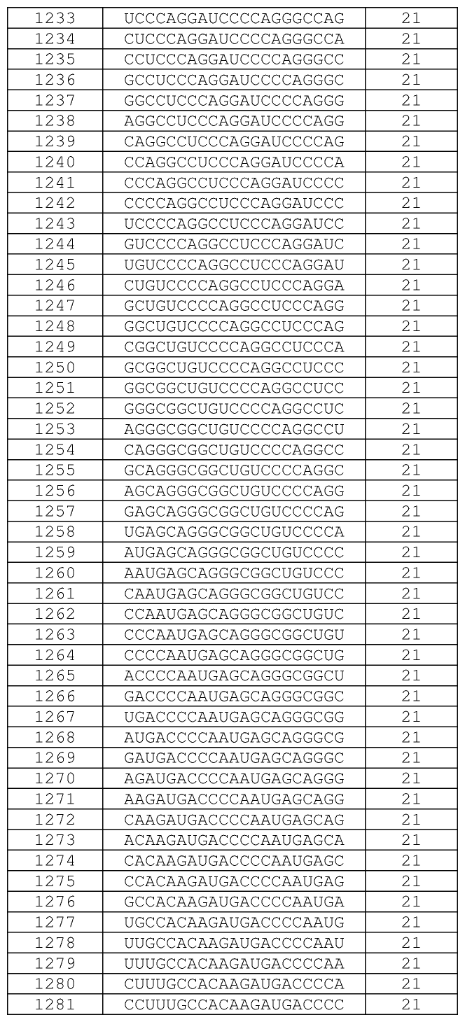

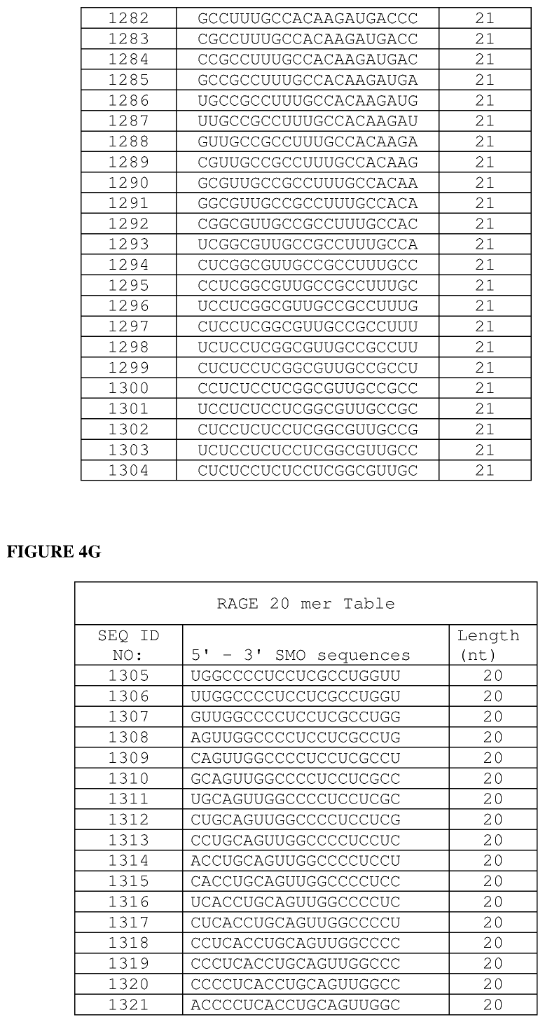

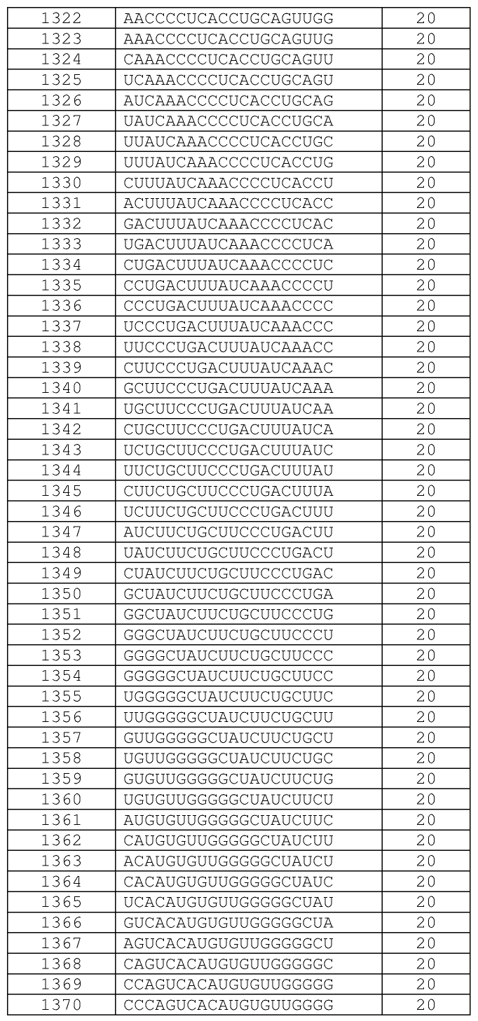

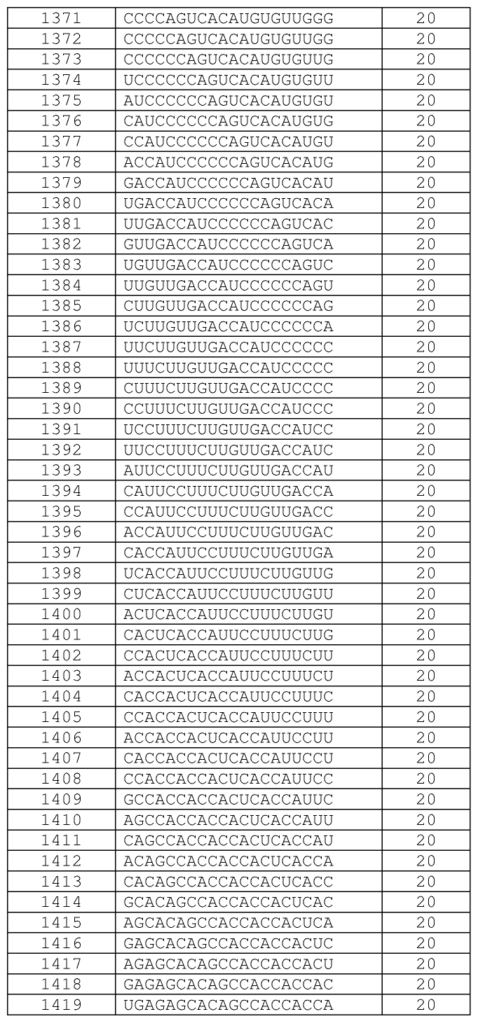

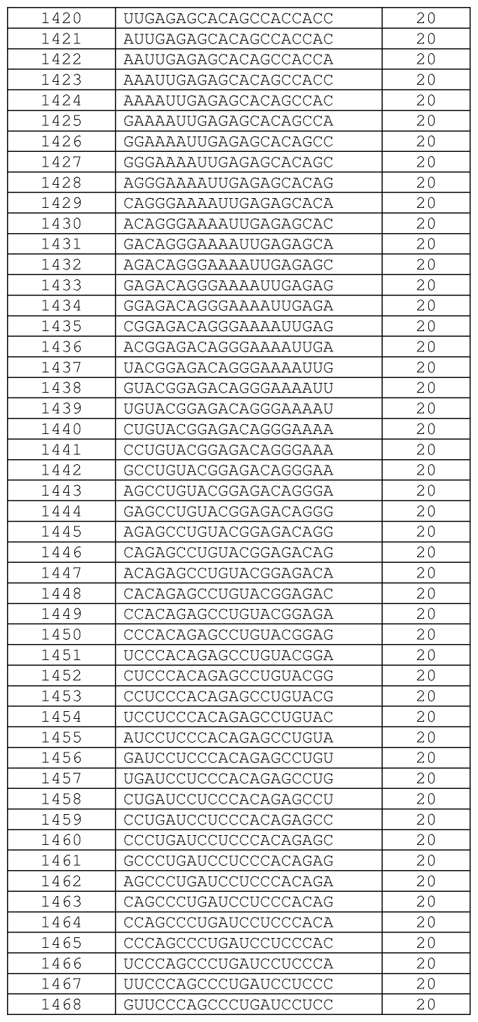

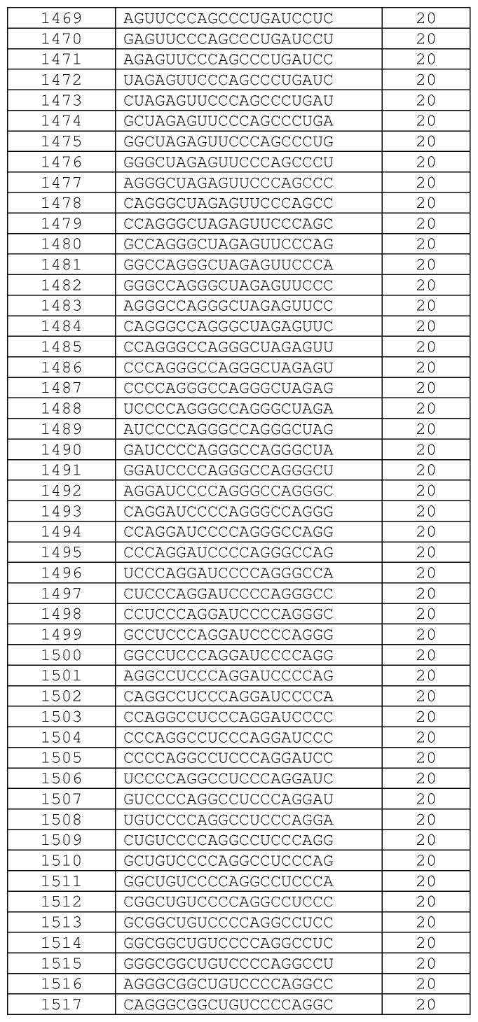

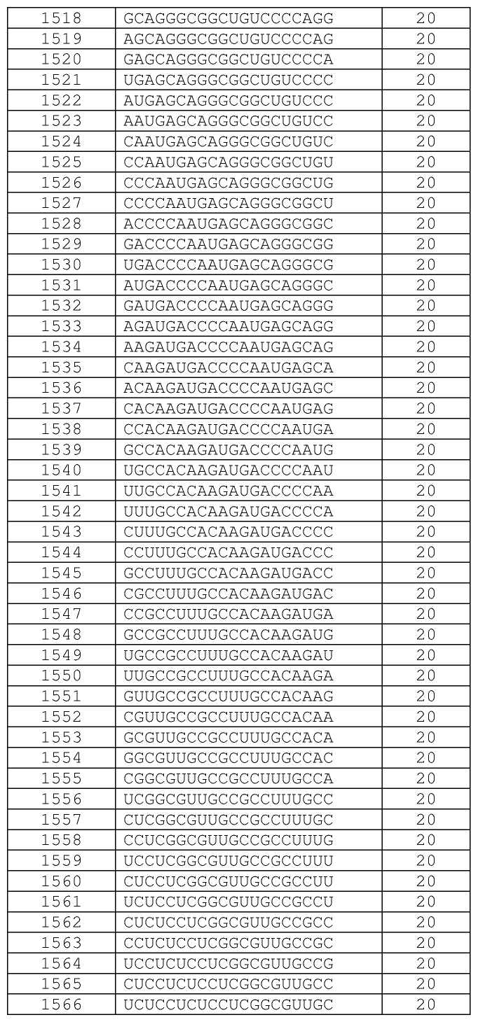

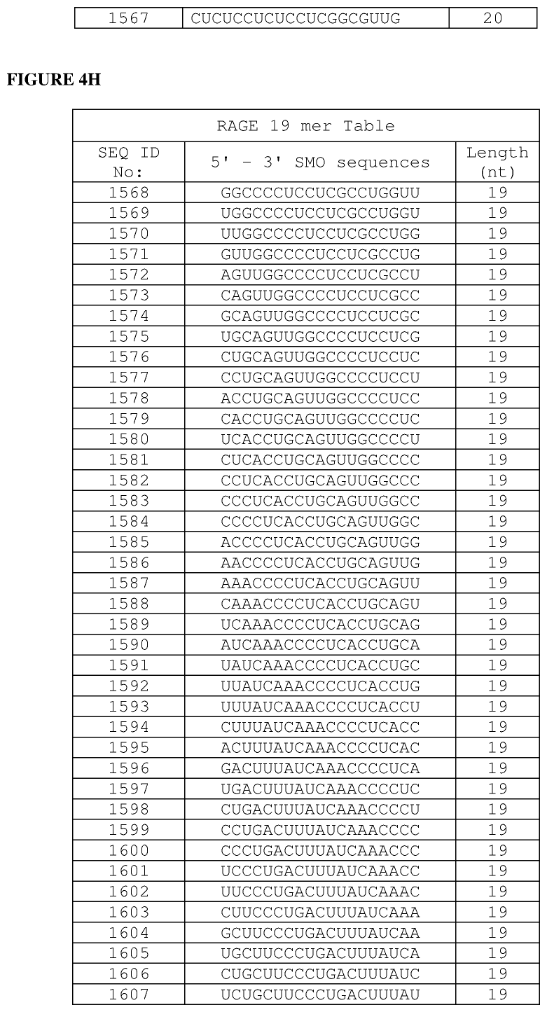

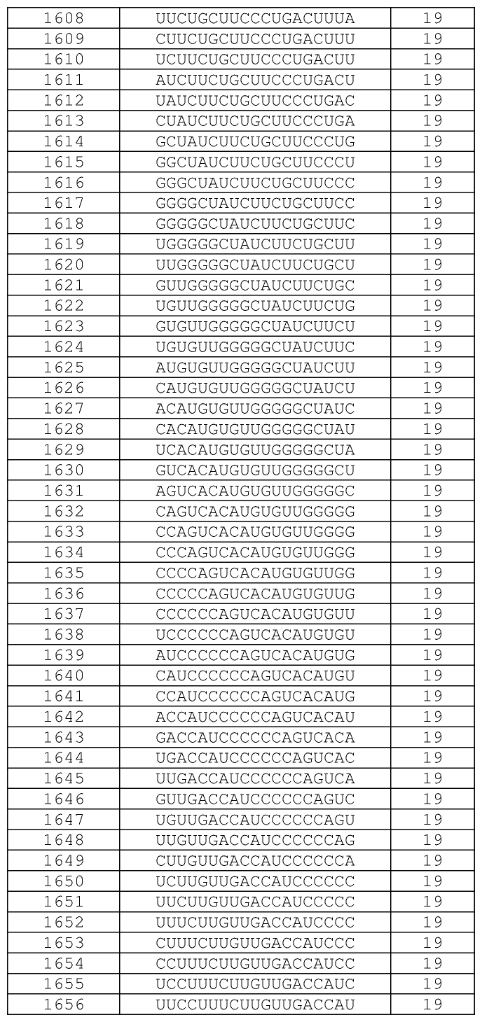

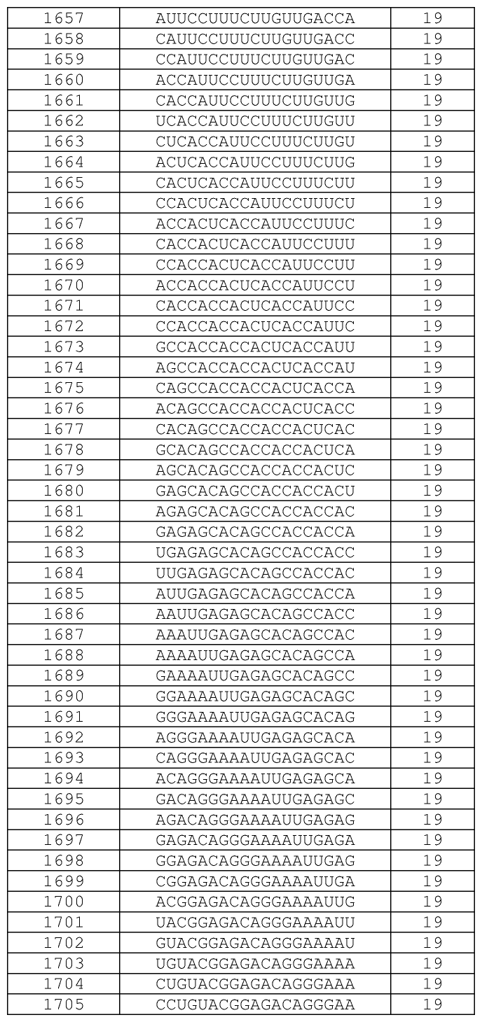

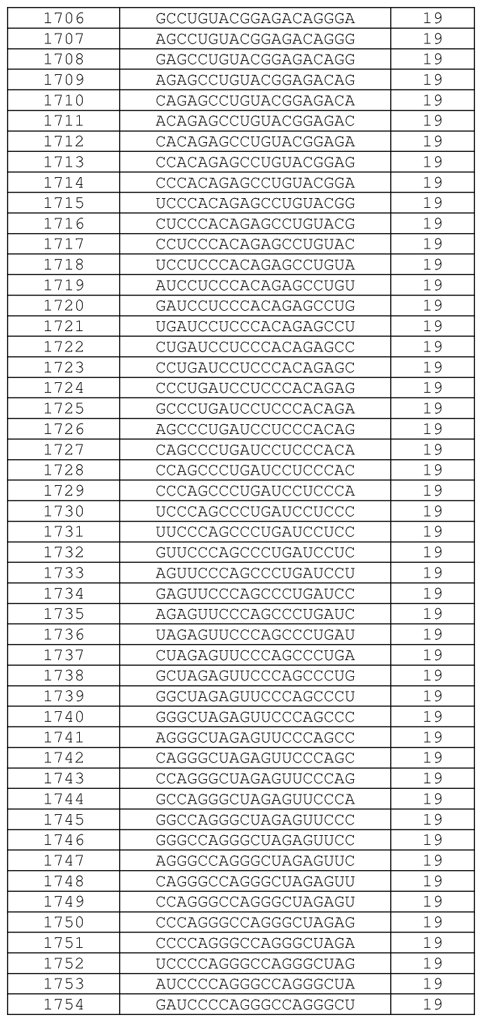

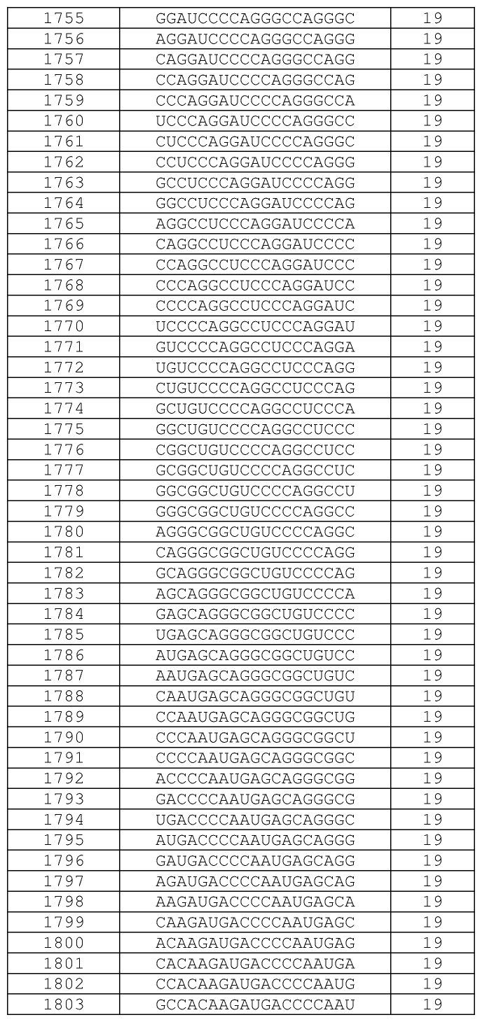

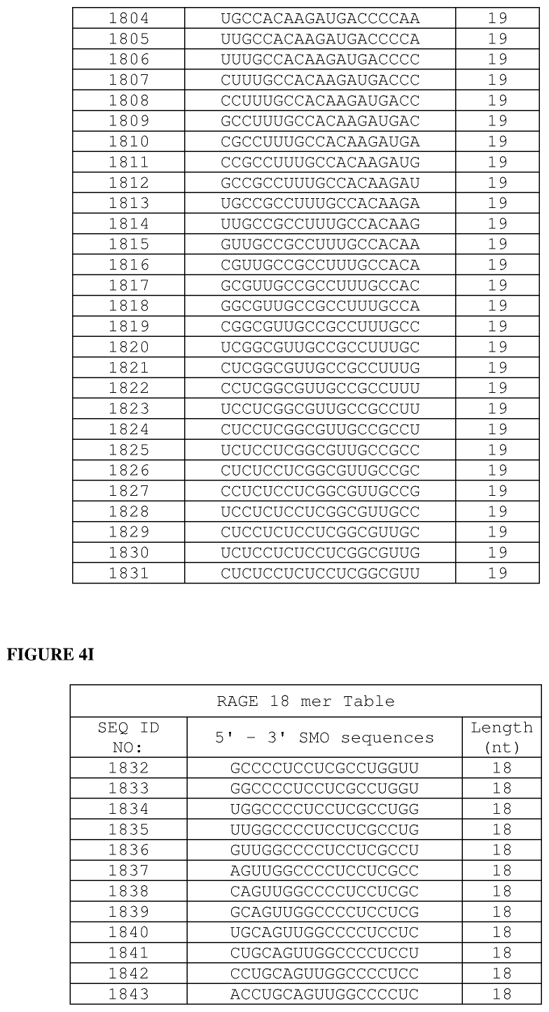

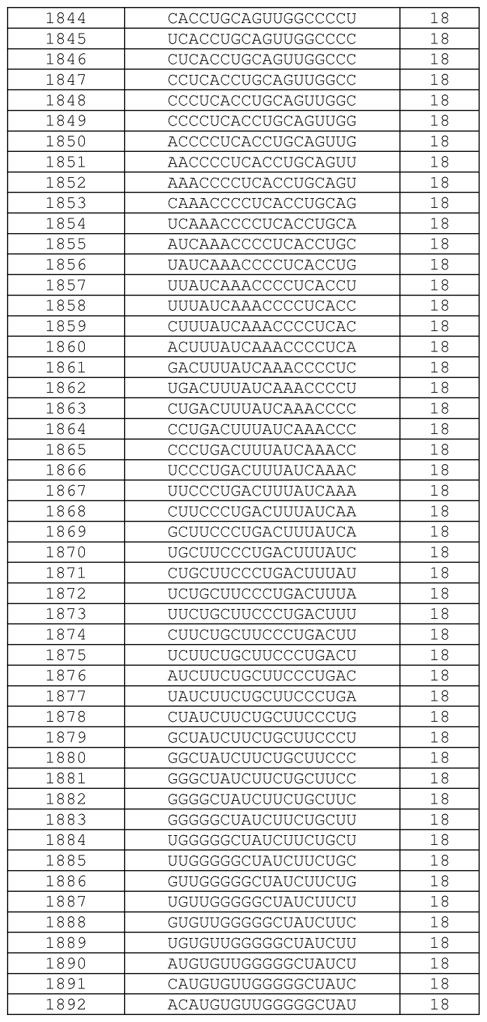

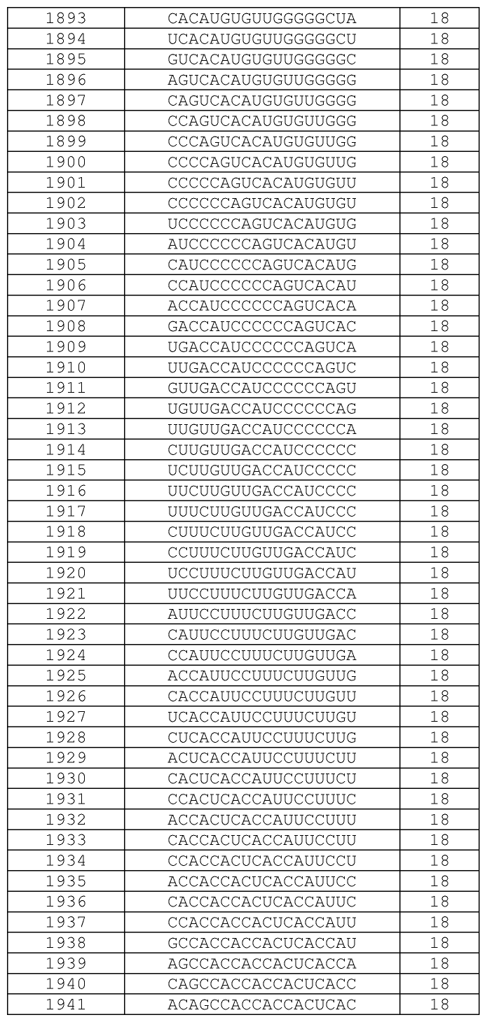

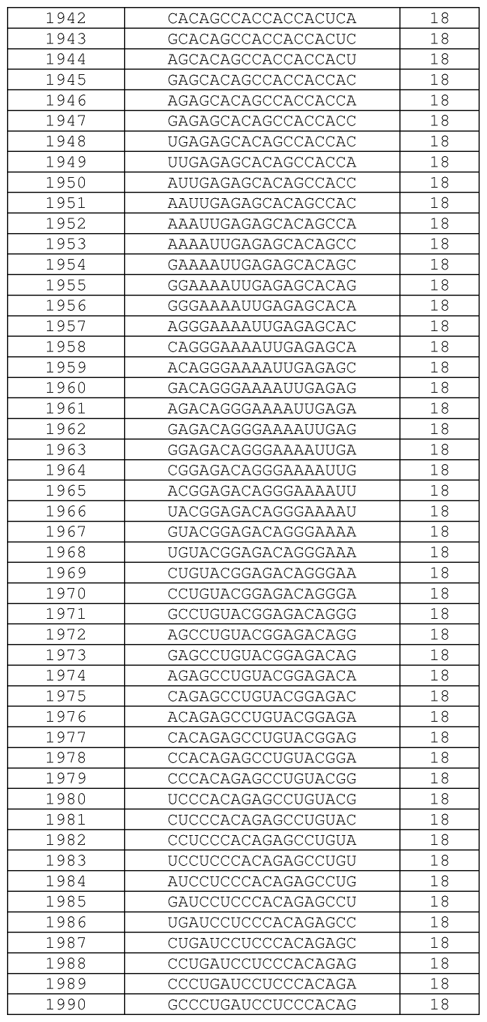

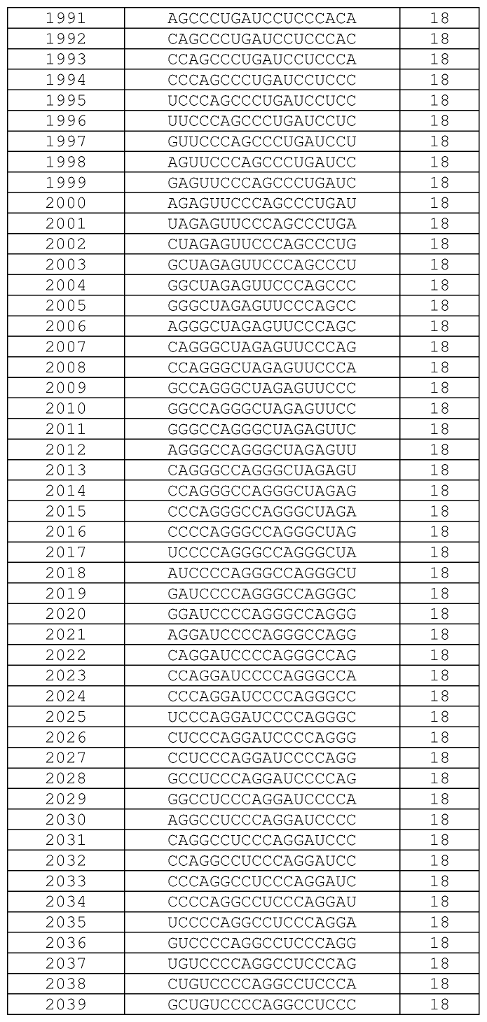

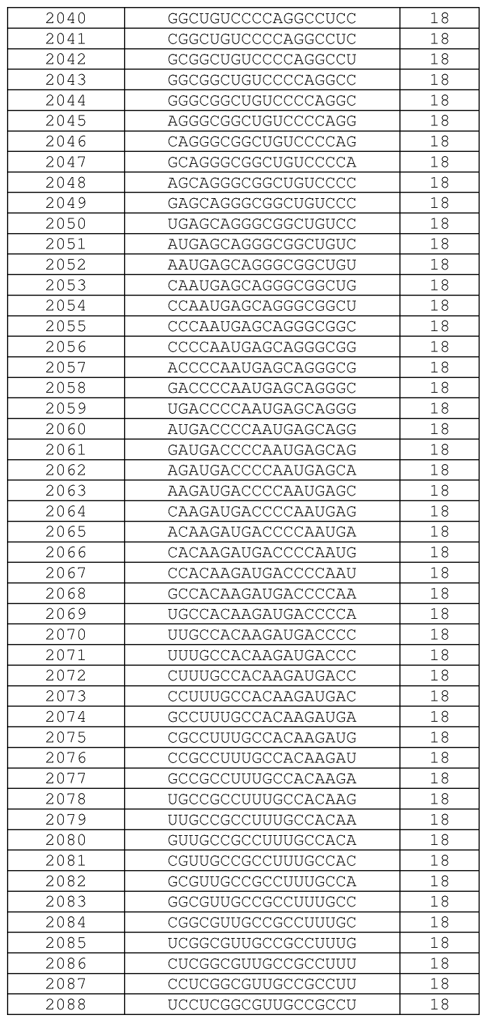

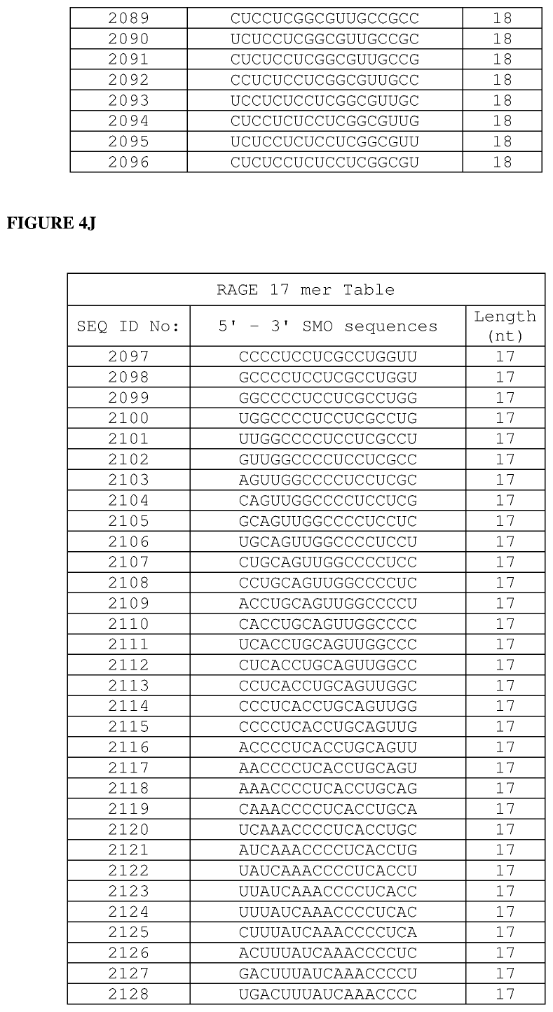

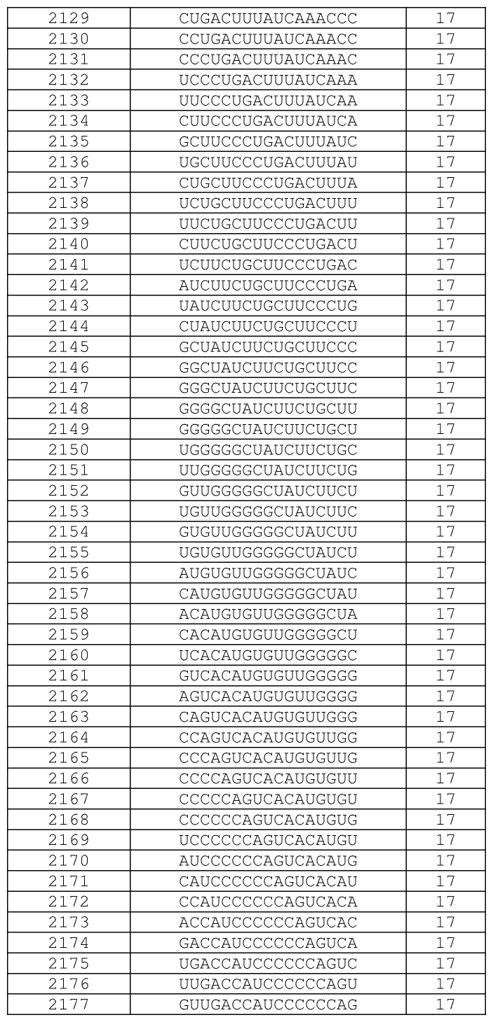

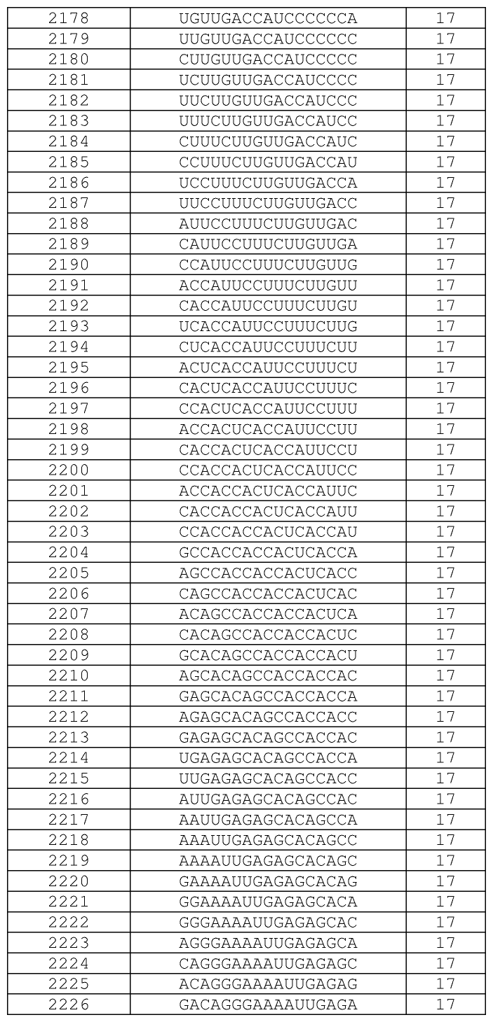

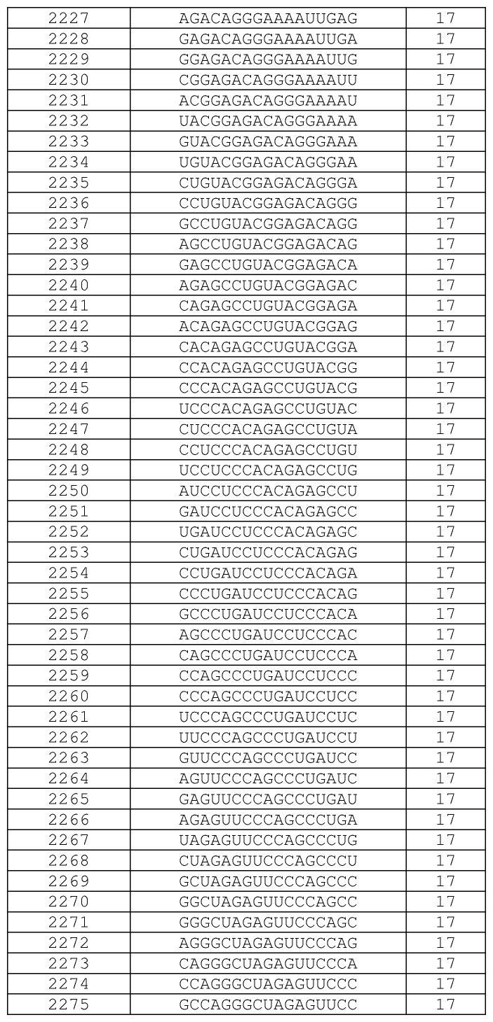

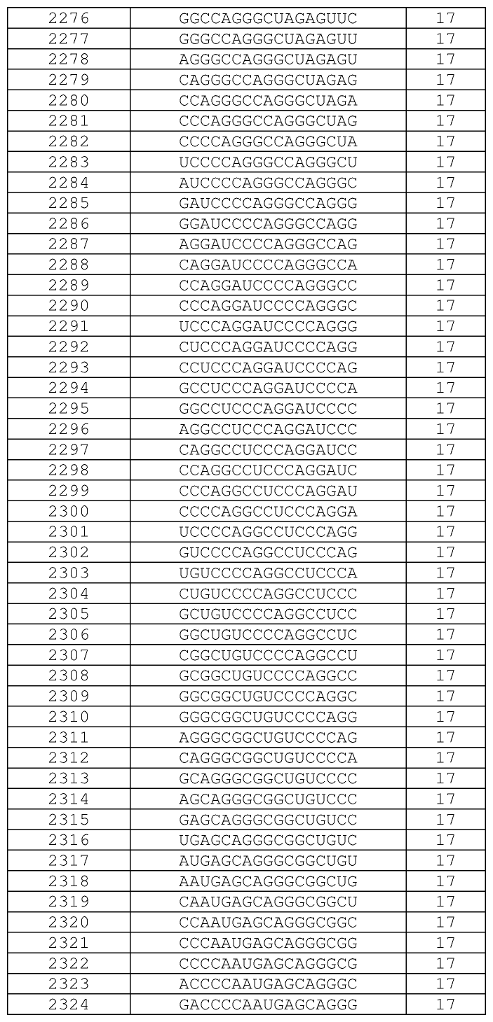

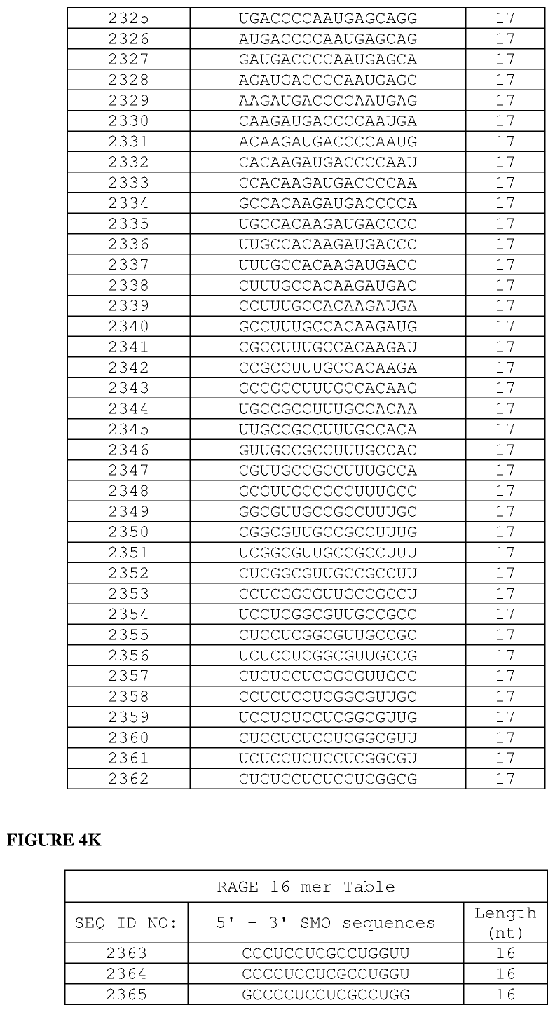

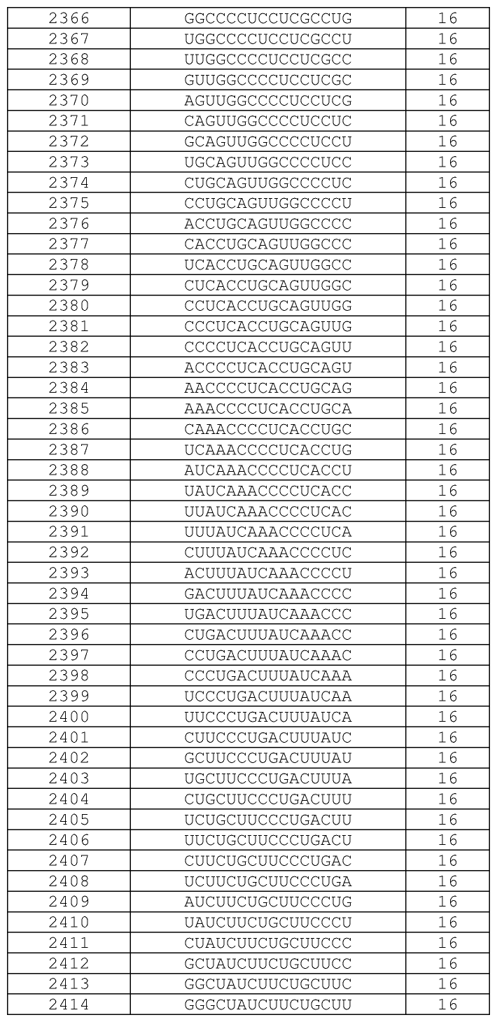

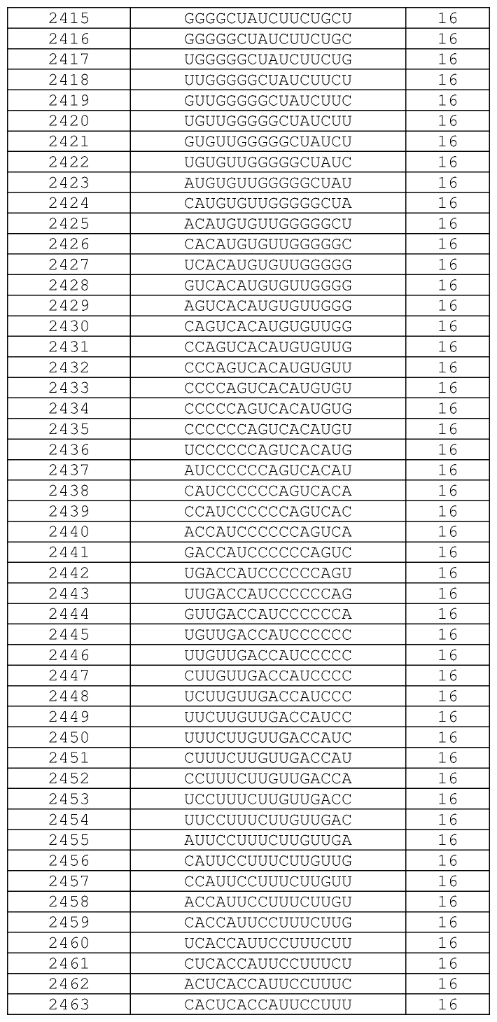

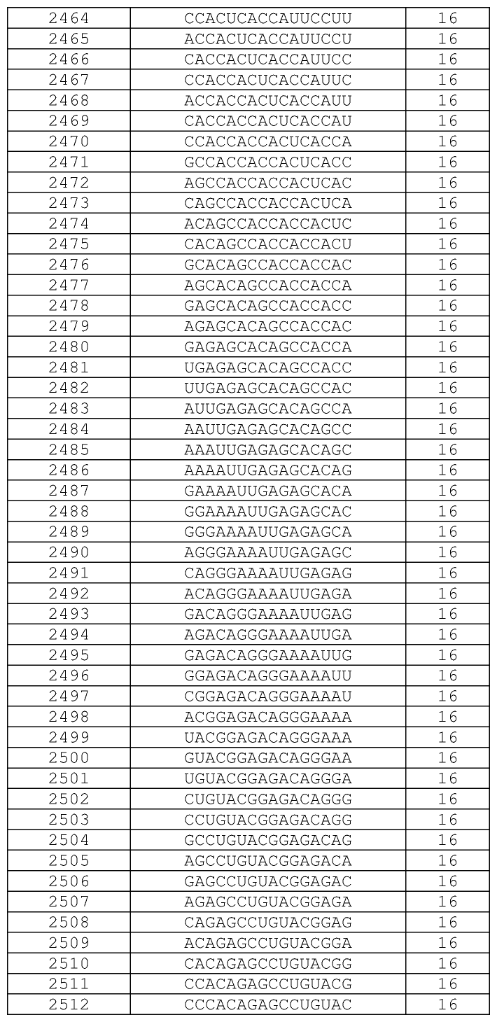

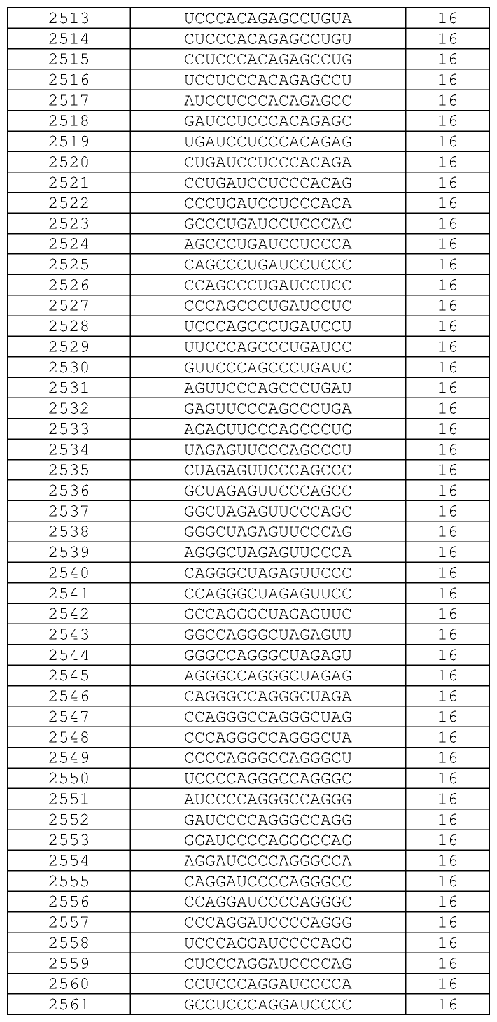

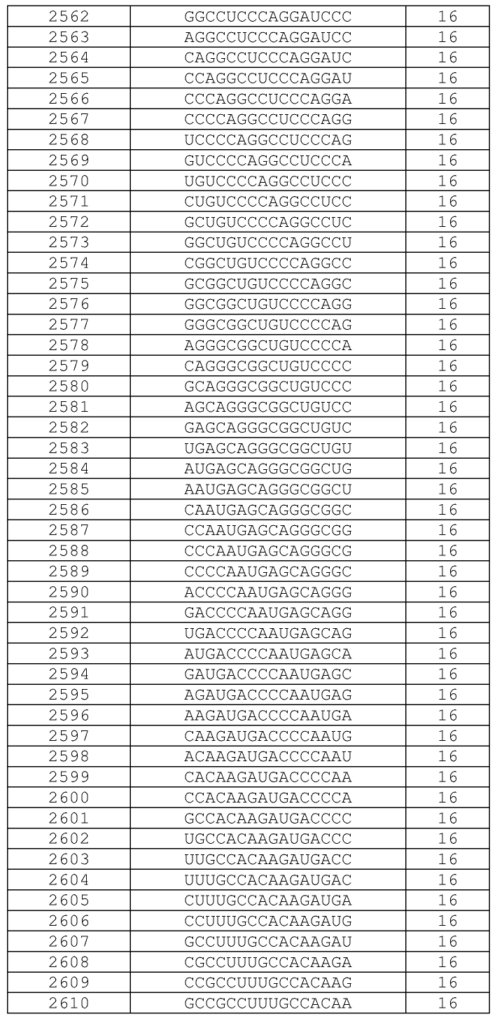

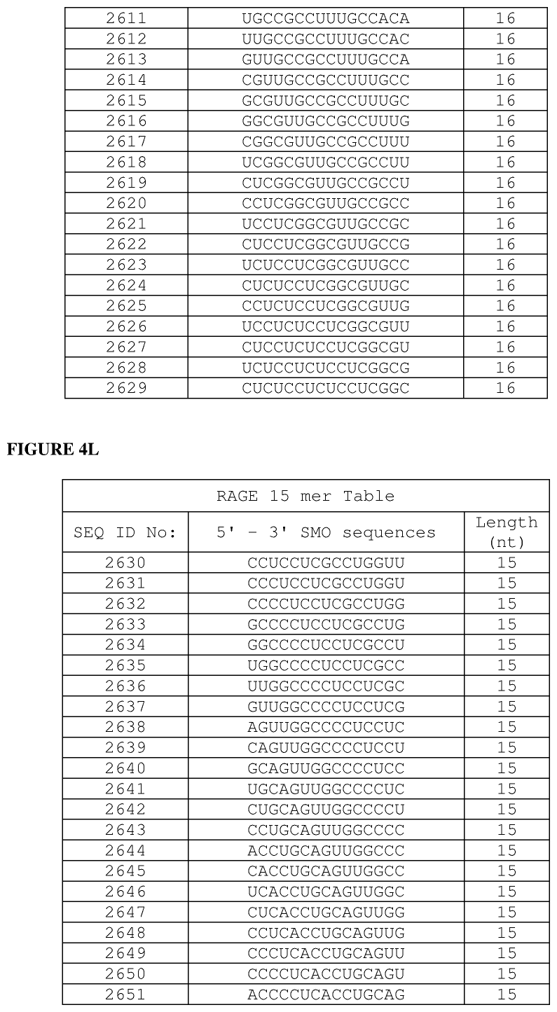

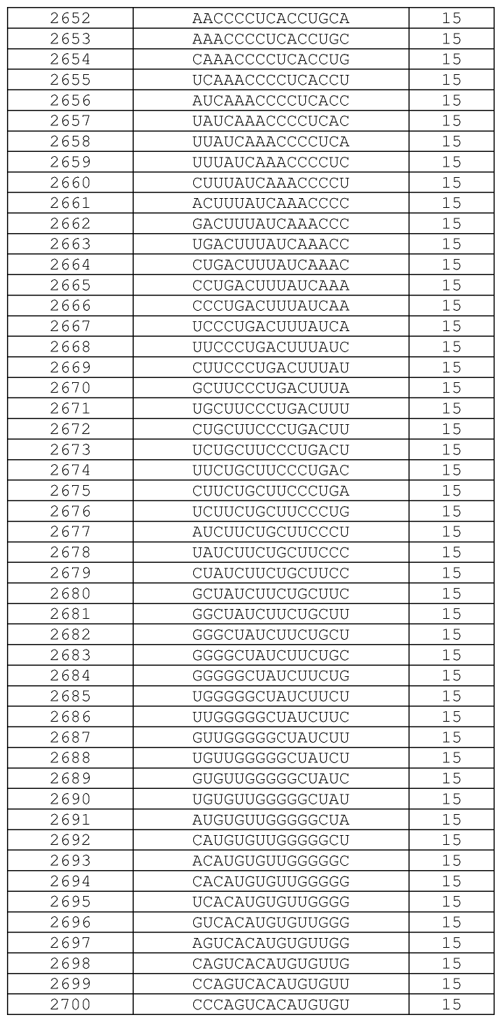

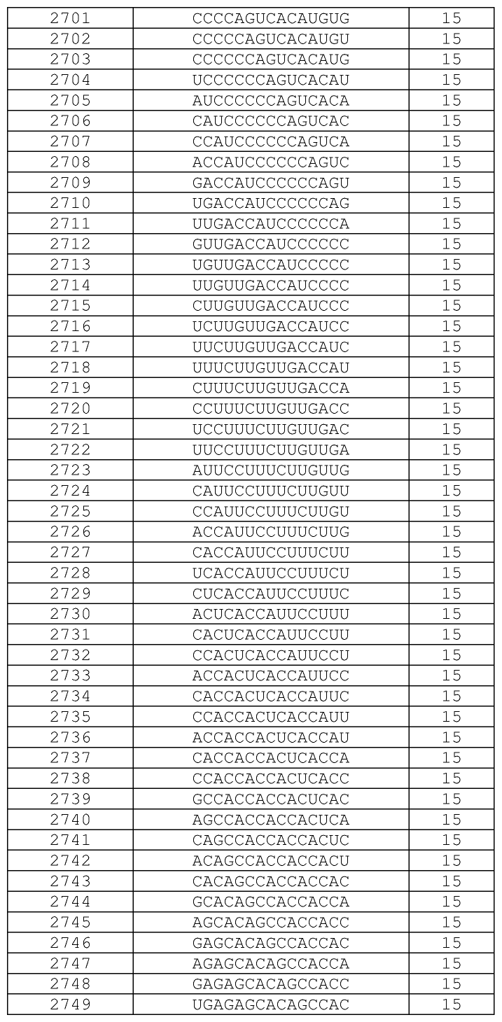

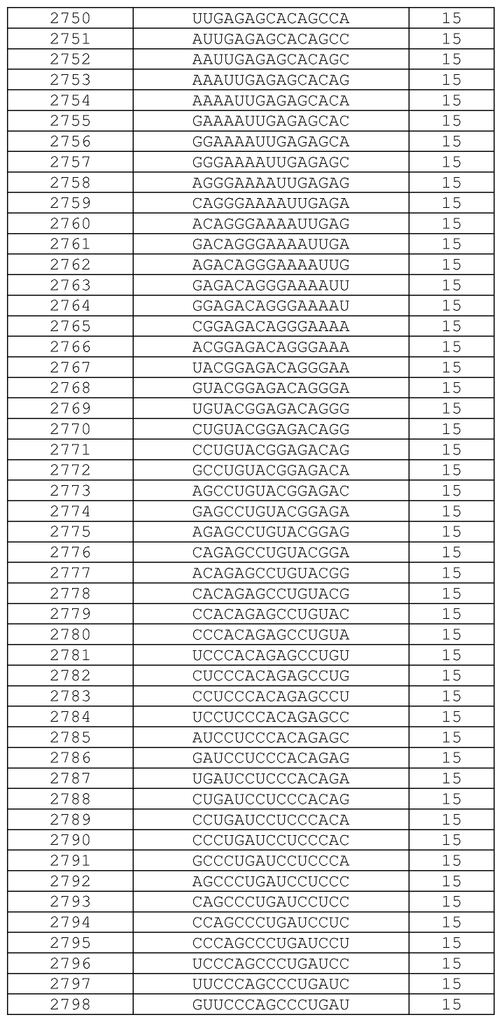

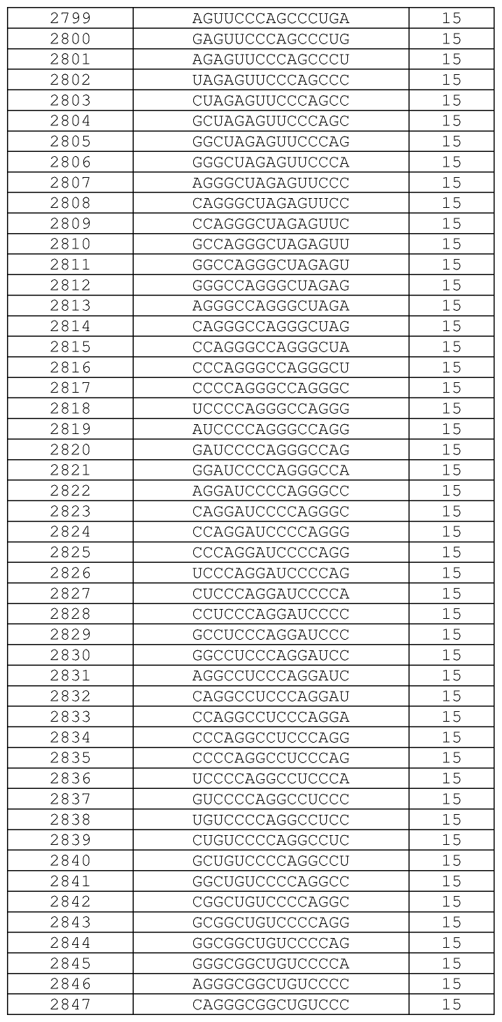

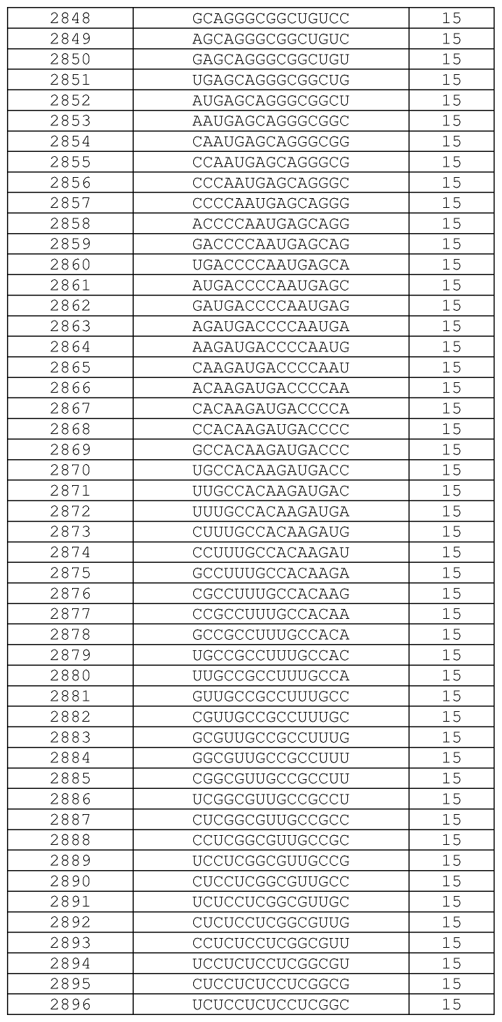

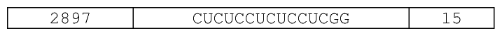

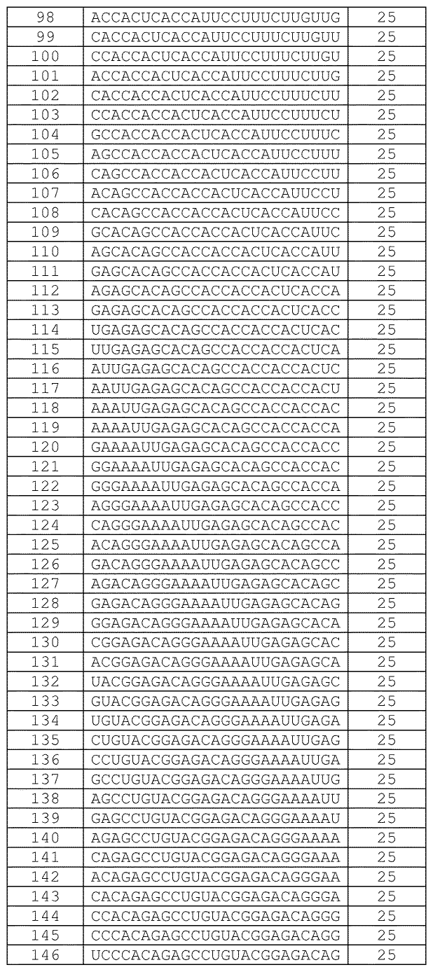

The invention provides compositions and methods for modulating RAGE pre-mRNA splicing using an SMO of the invention. For example, in various examples, an SMO may modulate pre-mRNA splicing by removing an exon (e.g., exon 10), including an exon (e.g., exon 9), or inducing full or partial inclusion of an intron (e.g., exon 9), in order to alter protein isoform expression (e.g., to enhance expression of sRAGE isoforms with decoy receptor function, or decrease expression of membrane bound RAGE isoforms with receptor signaling function, or a combination thereof). For example, an SMO as described herein may modulate RAGE pre-mRNA by read-through of the 5′ splice site of exon 9 resulting in inclusion of part or all of intron 9, or excluding exon 10, or any combination thereof in the resulting RAGE mRNA. These SMOs may be used to modify RAGE properties, i.e., to produce isoforms with decoy receptor function, or inhibit production of RAGE isoforms with receptor signaling function, or a combination thereof. In other embodiments, an SMO described herein may modulate RAGE pre-mRNA by read-through of the 5′ splice site of exon 9 resulting in inclusion of part or all of intron 9, or excluding exon 10, or any combination thereof in the resulting RAGE mRNA. These SMOs may be used to generate a RAGE protein that has decoy receptor function, or that is not translated. Details of possible splicing patterns obtained using the methods of the invention are set forth in FIG. 3.

Accordingly, certain embodiments of the invention provide a method of modulating splicing of a RAGE pre-mRNA, either in vitro or in vivo comprising contacting a cell with an effective amount of an SMO or composition described herein. In certain embodiments, the SMO specifically binds to a RAGE pre-mRNA sequence (e.g., at an intron/exon splice site, ESE and/or ISE), thereby causing read-through of the 5′ splice site of exon 9 resulting in inclusion of part or all of intron 9, or exclusion of exon 10, or any combination thereof from a resulting RAGE mRNA.

Certain embodiments of the invention provide a method of modulating splicing of a RAGE pre-mRNA comprising contacting a cell with an effective amount of an SMO that specifically binds to a complementary sequence on the pre-mRNA at a intron-exon splice site, ESE and/or ISE, wherein when the SMO specifically binds to the complementary sequence, causing read-through of the 5′ splice site of exon 9 resulting in inclusion of part or all of intron 9, or exclusion of exon 10, or any combination thereof in the resulting mRNA, and wherein the resulting mRNA encodes a RAGE protein.

Certain embodiments of the invention provide a method of modulating splicing of a RAGE pre-mRNA comprising contacting a cell with an effective amount of an SMO that specifically binds to a complementary sequence on the pre-mRNA at a intron-exon splice site, ESE and/or ISE, wherein when the SMO specifically binds to the complementary sequence, causing read-through of the 5′ splice site of exon 9 resulting in inclusion of part or all of intron 9, or exclusion of exon 10, or any combination thereof in the resulting mRNA, and wherein the resulting mRNA encodes a RAGE protein.

In some embodiments, sRAGE (including RAGEv1) protein production is enhanced in a treated cell, cell extract, organism or patient, with an enhancement of sRAGE (including RAGEv1) RAGE protein levels of at least about 1.1-, 1.2-, 1.5-, 2-, 3-, 4-, 5-, 7-, 10-, 20-, 100-fold and higher values being exemplary. In another embodiment of the invention, membrane bound RAGE (including flRAGE) protein production is reduced in a treated cell, cell extract, organism or patient, with a decrement of membrane bound RAGE (including flRAGE) protein levels of at least about 1.1-, 1.2-, 1.5-, 2-, 3-, 4-, 5-, 7-, 10-, 20-, 100-fold and lower values being exemplary. Enhancement of gene expression refers to the presence (or observable increase) in the level of protein and/or mRNA product from a target RNA. Decrement in gene expression refers to the absence (or observable decrease) in the level of protein and/or mRNA product from a target RNA. Specificity refers to the ability to act on the target RNA without manifest effects on other genes of the cell. The consequences of modulation of the target RNA can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

### Methods of Treating Diseases and Disorders

A wide range of different diseases and conditions are associated with increased flRAGE expression or activity (over activation), increased expression of RAGE ligands, and/or decreased sRAGE, or otherwise are associated with dysregulation of RAGE activity or function. The invention can thus be used to modulate RAGE pre-mRNA splicing to correct pathological RAGE activity or function caused by excess membrane bound RAGE (including flRAGE) or decreased sRAGE (including RAGEv1). Further RAGE pre-mRNA splicing can be modulated to treat any disease or disorder to which reducing membrane bound RAGE (including flRAGE) or increasing sRAGE (including RAGEv1) is therapeutic. RAGE pre-mRNA splicing is also modulated as a tool for studying RAGE both in vitro and in vivo. More generally, the invention can be used in the treatment or prevention of diseases and disorders in which dysregulation of RAGE ligands and RAGE isoform expression, including dysregulated RAGE alternative splicing, have been shown to contribute significantly to disease pathology.

For example, the invention can be used in the treatment of neurological diseases and conditions, including neurodegenerative diseases and conditions. Thus, in various examples, the invention can be used in the treatment of Alzheimer's disease, amyotropic lateral sclerosis (ALS), brain injury, and related neurological and neuroinflammatory diseases or conditions.

The invention can further be used in the treatment and prevention (e.g., prevention of recurrence and/or metastases) of cancer including, e.g., brain cancer (such as glioma or glioblastoma), lung cancer, prostate cancer, gastric cancer, colon cancer, common bile duct cancer, pancreatic cancer, breast cancer, liver cancer, and cancer-treatment-related pain.

In addition, the invention can be used to treat or prevent diabetes mellitus (type I or type II) and related diseases or conditions. For example, the invention can be used in the treatment or prevention of pre-diabetes, glucose intolerance, diabetic allodynia, neuropathy (e.g., peripheral neuropathy), diabetes-related atherosclerosis (including coronary artery disease and peripheral artery disease), diabetic peripheral vascular disease, diabetic ischemia, diabetic pain, diabetic retinopathy, diabetic nephropathy, and diabetic wound healing.

The invention can additionally be used in the treatment and prevention of pulmonary diseases and conditions including, e.g., respiratory distress syndrome (RDS), including acute RDS (ARDS), acute lung injury (ALI), chronic obstructive pulmonary disease, emphysema, asthma, cystic fibrosis, idiopathic pulmonary fibrosis, and airway injury.

Further, the invention can be used in the treatment and prevention of cardiovascular diseases and conditions including, e.g., atherosclerosis (including coronary artery disease and peripheral artery disease), heart failure, ischemia-reperfusion injury, and stroke.

The invention can also be used in the treatment and prevention of immunological, inflammatory, and autoimmune diseases including, e.g., lupus, multiple sclerosis, osteoarthritis, rheumatoid arthritis, sepsis, transplant rejection (e.g., heart, kidney, or islet cells), graft vs. host disease, and inflammatory bowel syndrome

The invention can also be used in the treatment and prevention of liver diseases and conditions including, e.g., non-alcoholic fatty liver disease (NAFLD), fibrosis, cirrhosis, hepatocellular carcinoma, hepatitis (e.g., hepatitis B), and liver fibrosis.

Nucleic acid molecules (e.g., SMOs) can be administered for use in the methods of the invention using methods that are known in the art. SMOs are typically administered to a subject, or generated in situ, such that they hybridize with or bind to RAGE pre-mRNA, as described above. The method of delivery selected will depend on factors including, e.g., the cells, tissues, or organs to be treated and their locations, as understood by those skilled in the art. Delivery can be systemic or targeted, with targeting optionally being achieved by the use of a targeting agent or by local administration.

In some examples, conjugation of an SMO to agents facilitating their delivery, e.g., anthraquinones, acridines, biotin, carbohydrates, chitosans, cholesterol, phospholipids, dendrimers, other lipid and liposomal moieties, colloidal polymeric particles, coumarins, dyes (such as fluoresceins and rhodamines), folate, peptides, phenanthridine, and phenazines, N-Acetylgalactosamine (GalNAc), other sugar derivatives, as well as other means known in the art, can be used to deliver the SMOs to cells.

In other examples, SMOs are delivered using one or more of, e.g., methods involving liposome-mediated uptake, lipid conjugates, sugar-derivative conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (see, e.g., Dokka et al., Adv. Drug De. Rev. 44(1):35-49, 2000; Winkler et al., Ther. Deliv. 4(7):791-809, 2013). Methods of delivery may also include the use of cationic lipids (e.g., N-[-1-(2,3-dioleoyloxy)propyl]N,N,N-triethylammonium chloride (DOTMA) and a 1:1 molar ratio of 1,2-dimyristyloxy-propyl-3-dimethylhydroxyethylammonium bromide (DMRIE) and dioleoyl phosphatidylethanolamine (DOPE); see e.g., Logan et al., Gene Therapy 2:38-49, 1995; San et al., Human Gene Therapy 4:781-788, 1993); receptor-mediated uptake (e.g., by complexing to a cation, such as polylysine, which is coupled to a ligand for a cell-surface receptor; see for example Wu et al., J. Biol. Chem. 263:14621, 1988; Wilson et al., J. Biol. Chem. 267:963-967, 1992; and U.S. Pat. No. 5,166,320); adenovirus capsids (see, for example, Curiel et al., Proc. Natl. Acad. Sci. USA 88:8850, 1991; Cristiano et al., Proc. Natl. Acad. Sci. U.S.A. 90:2122-2126, 1993); and lipid-based compounds that are not liposomes (e.g., lipofectins and cytofectins).

The SMOs can optionally be delivered by routes including, for example, intravenous, intramuscular, intradermal, intravitreous, subcutaneous, intranasal, and transdermal routes. Oligonucleotides, including SMOs, can be directly introduced into a cell, tissue, or organ, or introduced extracellularly into a cavity, interstitial space, or the circulation of a subject. Vascular or extravascular circulation, including the blood or lymph systems, and the cerebrospinal fluid, are examples of sites where an oligonucleotide, such as an SMO, may be introduced. In one embodiment, an SMO is delivered directly into the cerebral spinal fluid (CSF) of a subject, e.g., by epidural injection, intrathecal or intracerebroventricular injection (e.g., using an infusion pump), or direct brain delivery with a pump or other device.

SMOs can be modified to promote crossing of the blood-brain-barrier (BBB) to achieve their delivery to the central nervous system (CNS; see, e.g., Forte et al., Curr. Drug Targets 6:21-29, 2005; Jaeger et al., Methods Mol. Med. 106:237-251, 2005; Vinogradov et al., Bioconjug. Chem. 5:50-60, 2004). In some examples, SMOs are conjugated to a peptide to facilitate delivery of the SMO across the following parenteral administration to a subject. The SMO can be either directly conjugated to the peptide or indirectly conjugated to the peptide via a linker molecule, such as a poly amino acid linker, or by electrostatic interaction. Peptides useful in delivering SMOs across the BBB include, e.g., peptides derived from the rabies virus glycoprotein (RVG) that specifically bind to the nicotinic acetylcholine receptor (AchR) present on neurons and the vascular endothelium of the BBB, thereby allowing transvascular delivery, probably by receptor-mediated transcytosis (Kumar et al., Nature 448:39-43, 2007); Kunitz domain-derived peptides called angiopeps (Demeule et al., J. Neurochem. 106: 1534-1544, 2008; Demeule et al., J. Pharmacol. Exp. Ther. 324:1064-1072, 2008).

Recombinant methods known in the art can also be used to achieve oligonucleotide reagent-induced modulation of splicing in a target nucleic acid. For example, vectors containing oligonucleotides can be employed to express, e.g., an antisense oligonucleotide to modulate splicing of an exon of a targeted pre-mRNA.

SMOs can be administered in doses and in regimens determined to be appropriate by those of skill in the art. For example, dosing for CNS manifestations can be accomplished by direct bolus intrathecal injection as infrequently as every 1-6 months, weekly in multiple loading doses, or by continuous infusion via pump (i.e., Omaya Reservoir) directly into the hippocampus. Dosing for peripheral indications can be achieved through subcutaneous or intravenous injections as infrequently as every 1-6 months, or a multiple loading dose strategy could also be used.

### Pharmaceutical Compositions and Therapies

The invention also provides pharmaceutical compositions including one or more SMO of the invention, optionally in combination with a pharmaceutically-acceptable carrier or diluent (e.g., sterile isotonic saline or sterile water). The compositions may be in the form of a liquid or may be in dried form. As used herein the term “pharmaceutically acceptable carrier” is intended to include any and all solvents, dispersion media, coatings, antibacterial and antifungal agents, isotonic, and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Standard formulations that can be used in the invention are described, e.g., in Remington's Pharmaceutical Sciences (1985, Genaro, ed., Mack Publishing Co., Easton, Pa.).

The oligonucleotide, i.e. the SMO, may be introduced in an amount which allows delivery of at least one copy per cell. Higher doses (e.g., at least 5, 10, 100, 500 or 1000 copies per cell) of material may yield more effective modulation; lower doses may also be useful for specific applications.

The therapeutic and prophylactic methods of the invention thus encompass the use of pharmaceutical compositions comprising a splice modifying oligonucleotide of the invention to practice the methods of the invention. The precise dosage administered will vary depending upon any number of factors, including but not limited to, the type of animal and type of disease state being treated, the age of the animal and the route of administration.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

### Kits

The invention further provides kits for practicing the methods of the invention. By a “kit” is intended any manufacture (e.g., a package or a container) including at least one reagent, e.g., at least one SMO, for targeting RAGE, as described herein, and for the treatment or prevention of a disease, disorder, or condition, e.g., Alzheimer's disease (also see above). In one embodiment, the kit includes at least one SMO for specifically enhancing the expression of sRAGE (including RAGEv1) protein, reducing membrane bound RAGE (including flRAGE), or a combination thereof (e.g., for enhancing the read-through the 5′ splice site of exon 9 resulting in inclusion of part or all of intron 9, or exclusion of exon 10, or a combination thereof). The kits may contain a package insert describing the kit and including instructional material for its use. Further, positive, negative, and/or comparator controls may be included in the kits.

### Animal Models and Screening Methods

The invention also includes animal models that can be used to identify or characterize SMOs directed against RAGE, such as those of the invention. In various embodiments, the animal models are mice (e.g., C57/B6 mice) that express human RAGE (RAGE Tg mice). The animal models can be transgenic animals, in which a human AGER sequence is introduced into the genome of the animal, such that it is capable of producing alternatively spliced RAGE mRNA variants. The human AGER sequence can optionally be introduced into the genome in place of or in addition to the AGER sequence of the animal using methods that are known in the art. For example, methods including the use of CRISPR/Cas-9 or another gene editing approach can be used. Additionally, approaches utilizing standard homologous recombination or microinjection of modified ES cells can be used.

In some embodiments, the animal model is a mouse and, optionally, the mouse sequence encoding RAGE is deleted from the genome of the mouse using, e.g., gene editing methods (e.g., CRISPR/Cas9-based methods). In some embodiments, a human sequence encoding RAGE, such as a genomic sequence encoding exons and introns (e.g., NCBI Reference Sequence: NM_001206929.1), is inserted into the genome of the mouse so that it is under the control of the mouse promoter that directs expression of RAGE.

In one example, the RAGE-Tg mouse is a humanized model, whereby the human AGER gene containing both exons and introns was replaced in C57BL/6 mice under the control of the mouse RAGE promoter using CRISPR/Cas-mediated genome engineering. Mouse Ager (ATG start codon to TAA stop codon) was replaced with the human AGER (ATG start codon to TGA stop codon) cassette. The human AGER gene (NCBI Reference Sequence: NM_001206929.1), located on human chromosome 6 contains eleven exons, with the ATG start codon in exon 1 and TGA stop codon in exon 11. The mouse Ager gene (NCBI Reference Sequence: NM_007425.3), located on mouse chromosome 17, also contains eleven exons have been identified, but with the ATG start codon in exon 1 and TAA stop codon in exon 11. To engineer the donor vector, homology arms were generated by PCR using BAC clone RP24-357H14 and RP24-376H18 from the C57BL/6 library as template. Cas9 and guide (g)RNA were co-injected into fertilized eggs with donor vector for mouse production.

### Guide RNA:

gRNA1 (matching reverse strand of gene):
(SEQ ID: 2903)
AGCTGCTGTCCCCGCTGGCATGG
gRNA2 (matching reverse strand of gene):
(SED ID: 2904)
TGGGTGCTCTTACGGTCCCCCGG

The resulting F0 pups were genotyped by PCR with gel electrophoresis confirmation of the product size, followed by Sanger sequencing of PCR product. Three F0 mice with targeted insertion of the humanized AGER gene (RAGE-Tg) were then bred to C57BL/6 mice to generate F1 mice, and so forth.

The animal models of the invention can be used in methods to identify or characterize SMOs directed against human RAGE. For example, an SMO directed against human RAGE (e.g., an SMO comprising, consisting essentially of, or consisting of a sequence of SEQ ID NO: 5-2897) can be introduced into an animal model (e.g., a neonatal RAGE transgenic mouse) and the effects of this treatment monitored. In various embodiments, effects on splicing are monitored. For example, expression of membrane bound RAGE (including flRAGE) and sRAGE (including RAGEv1) is evaluated, e.g., by RT-QPCR. In another example, SMOs are tested in an inducible model disease (e.g., an ICV STZ model of sporadic AD; see below) and effects on disease process, progression, cognition, or histopathology are examined. In yet another example, SMOs are tested in a model where RAGE Tg mice are bred to mice harboring disease-related mutations (e.g., presenilin mutations implicated in AD, SOD1 mutations implicated in ALS, or CFTR mutations implicated in cystic fibrosis) to generate mice harboring both the human RAGE transgene and disease mutation, and where effects on disease process, progression, cognition, or histopathology are examined.

### General Terminology

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are well known and commonly employed in the art.

Standard techniques are used for nucleic acid and peptide synthesis. The techniques and procedures are generally performed according to conventional methods in the art and various general references (e.g., Sambrook et al., 2001, Molecular Cloning, A Laboratory Approach, Cold Spring Harbor Press, Cold Spring Harbor, N.Y., and Ausubel et al., 2002, Current Protocols in Molecular Biology, John Wiley & Sons, NY), which are provided throughout this document.

The nomenclature used herein and the laboratory procedures used in analytical chemistry and organic syntheses described below are well known and commonly employed in the art. Standard techniques or modifications thereof, are used for chemical syntheses and chemical analyses.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used.

“Antisense activity” means any detectable or measurable activity attributable to the hybridization of an antisense compound to its target nucleic acid. In certain embodiments, antisense activity is a change in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as SMOs, antisense oligonucleotides, siRNAs, shRNAs, ssRNAs, and occupancy-based compounds. Antisense mechanisms include, without limitation, RNase H mediated antisense; RNAi mechanisms, which utilize the RISC pathway and include, without limitation, siRNA, ssRNA and microRNA mechanisms; and occupancy/steric block based mechanisms, including, without limitation uniform modified oligonucleotides. Certain antisense compounds may act through more than one such mechanism and/or through additional mechanisms.

“Antisense oligonucleotide” means a single-stranded oligonucleotide having a nucleobase sequence that permits hybridization or binding to a corresponding segment of a target nucleic acid.

A “disease” is a state of health of subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health. In some embodiments, the subject is an animal (e.g., a mammal, such as a human).

A disease or disorder is “alleviated” if the severity of a symptom of the disease or disorder, or the frequency with which such a symptom is experienced by a subject, or both, is reduced.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation. SMOs are administered in effective amounts, according to the methods of the invention.

As used herein “endogenous” refers to any material from or produced inside an organism, cell, tissue or system. As used herein, the term “exogenous” refers to any material introduced from or produced outside an organism, cell, tissue or system.

The term “expression” as used herein is defined as the transcription and/or translation of a particular nucleotide sequence. The term “exonic regulatory elements” as used herein refers to sequences present in pre-mRNA that enhance or suppress splicing of an exon. An exonic regulatory element that enhances splicing of an exon is an exonic splicing enhancer (ESE). An exonic regulatory element that suppresses splicing of an exon is an exonic splicing suppressor (ESS). An intronic regulatory element that enhances splicing of an exon is an intronic splicing enhancer (ISE). An intronic regulatory element that suppresses splicing of an exon is called an intronic splicing suppressor (ISS).

“Instructional material,” as used herein, includes a publication, a recording, a diagram, or any other medium of expression which can be used to communicate the usefulness of the composition and/or compound of the invention in a kit. It may, for example, be affixed to a container that contains a compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term also includes other modified nucleic acids as described herein. The term nucleic acid also specifically includes nucleic acids composed of bases other than the five biologically occurring bases (adenine, guanine, thymine, cytosine and uracil).

Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA which can be single- or double-stranded, optionally containing synthetic, non-natural, or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene, e.g., genomic DNA, and even synthetic DNA sequences. The term also includes sequences that include any of the known base analogs of DNA and RNA.

“Messenger RNA” or “mRNA” is any RNA that specifies the order of amino acids in a protein. It is produced by transcription of DNA by RNA polymerase. In eukaryotes, the initial RNA product (primary transcript, including introns and exons) undergoes processing to yield a functional mRNA (i.e., a mature mRNA), which is then transported to the cytoplasm for translation. “Precursor mRNA” or “pre-mRNA” includes the primary transcript and RNA processing intermediates; the spliceosome assembles on a pre-mRNA and carries out RNA splicing.

By “fragment” or “portion” is meant a full length or less than full length of the nucleotide sequence.

“Homology” refers to the percent identity between two polynucleotides or two polypeptide sequences. Two DNA or polypeptide sequences are “homologous” to each other when the sequences exhibit at least about 60% to 85% (including 65%, 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions.

The terms splice variant and splice isoform may be used interchangeably to denote different mRNAs, a product of which may or may not encode the same protein, but are a result of differential splicing from the same initial pre-mRNA sequence. Specifically, RAGE read-through the 5′ splice site of exon 9 resulting in inclusion of part or all of intron 9, or exclusion of exon 10, or a combination thereof generates the sRAGE (including RAGEv1) mRNA transcript variants, while read-through the 5′ splice site of exon 9 resulting in inclusion or part or all of intron 9, or exclusion of exon 10 also prevents generation of the membrane bound RAGE (including flRAGE) mRNA transcript variants. Generally, nucleotide sequence variants of the invention will have in at least one embodiment 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

The terms “isolated and/or purified” refer to in vitro isolation of a nucleic acid, e.g., a DNA or RNA molecule from its natural cellular environment, and from association with other components of the cell or test solution (e.g., RNA pool), such as nucleic acid or polypeptide, so that it can be sequenced, replicated, and/or expressed. Thus, the RNA or DNA is “isolated” in that it is free from at least one contaminating nucleic acid with which it is normally associated in the natural source of the RNA or DNA and is typically substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell, e.g., in a vector or plasmid.

Nucleic acid molecules having base substitutions (i.e., variants) are prepared by a variety of methods known in the art. These methods include, but are not limited to, isolation from a natural source (in the case of naturally occurring sequence variants) or preparation by oligonucleotide-mediated (or site-directed) mutagenesis, PCR mutagenesis, and cassette mutagenesis of an earlier prepared variant or a non-variant version of the nucleic acid molecule.

As used herein, the terms “derived” or “directed to” with respect to a nucleotide molecule means that the molecule has complementary sequence identity to a particular molecule of interest.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand;” sequences on the DNA strand which are located 5′ to a reference point on the DNA are referred to as “upstream sequences;” sequences on the DNA strand which are 3′ to a reference point on the DNA are referred to as “downstream sequences.”

The terms “protein,” “peptide,” and “polypeptide” are used interchangeably herein.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, e.g., Kunkel, Proc. Natl. Acad. Sci. U.S.A. 82:488, 1985; Kunkel et al., Meth. Enzymol., 154:367, 1987; U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, may be preferred.

“Polypeptide” also refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer. The term “protein” typically refers to large polypeptides.

The term “peptide” typically refers to short polypeptides. Conventional notation is used herein to portray polypeptide sequences: the left-hand end of a polypeptide sequence is the amino-terminus; the right-hand end of a polypeptide sequence is the carboxyl-terminus.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine. Polynucleotides can optionally include one or more modifications, analogs, and/or modified nucleotides, such as those described herein.

The term “oligonucleotide” typically refers to short polynucleotides, generally no greater than about 200 (e.g., up to 150, 100, 75, 60, 50, or 40) nucleotides. It will be understood that when a nucleotide sequence is represented by a DNA sequence (i.e., A, T, G, C), this also includes an RNA sequence (i.e., A, U, G, C) in which “U” replaces “T.” The term “recombinant DNA” as used herein is defined as DNA produced by joining pieces of DNA from different sources.

The term “recombinant polypeptide” as used herein is defined as a polypeptide produced by using recombinant DNA methods.

By the term “specifically binds,” as used herein, is meant a molecule, such as an SMO, which recognizes and binds to another molecule or feature (i.e., the target pre-mRNA), but does not substantially recognize or bind other molecules or features in a sample (i.e., other non-target pre-mRNAs). Two nucleic acids substantially recognize or bind to each other when at least about 50%, for example at least about 60% or at least about 80% of corresponding positions in each of the molecules are occupied by nucleotides which normally base pair with each other (e.g., A:T, A:U and G:C nucleotide pairs). For example, two nucleic acids substantially recognize or bind to each other when at least about 90%-100% of corresponding positions in each of the molecules are occupied by nucleotides which normally base pair with each other (e.g., A:T, A:U and G:C nucleotide pairs). Chemical modification of the nucleic acid in part determines hybridization energy and thus how many base pairs are required for specific binding of the SMO nucleic acid sequence and a target nucleic acid sequences. Such calculations are well within the ability of those skilled in the art.

The term “treatment,” as used herein, refers to reversing, alleviating, delaying the onset of, inhibiting the progress of, and/or preventing a disease or disorder, or one or more symptoms thereof, to which the term is applied in a subject. In some embodiments, treatment may be applied after one or more symptoms have developed. In other embodiments, treatment may be administered in the absence of symptoms. For example, treatment may be administered prior to symptoms (e.g., in light of a history of symptoms and/or one or more other susceptibility factors), or after symptoms have resolved, for example, to prevent or delay their reoccurrence.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

The invention will now be illustrated by the following non-limiting Example(s).

### Example

As described herein, SMOs were designed to specifically and potently direct selected alternatively spliced introns and exons in RAGE pre-mRNA and the efficacy of these SMOs is subsequently validated in transgenic mice expressing human RAGE and mouse models of Alzheimer's disease.

### SMOs for Targeting RAGE

RAGE is implicated in the pathogenesis of a number of diseases including, for example, Alzheimer's disease (AD). SMOs are developed to direct RAGE alternative splicing to decrease membrane bound-RAGE (including flRAGE), increase sRAGE (including RAGEv1), or a combination thereof, as described herein. In particular, SMOs are designed to impart a dual mechanism such that expression of membrane-bound (mbRAGE) or full length (flRAGE) RAGE is decreased, while concomitantly sRAGE is increased, both to reduce pathological RAGE signaling directly and clear RAGE-ligands (which also have other non-RAGE mechanisms of inducing damage) from systemic and CNS circulation.

### Design of Splice Modulating Oligonucleotides (SMOs)

Splice modulating oligonucleotides (SMOs) are designed and validated that specifically and potently modulate RAGE pre-mRNA splicing to decrease expression of the membrane bound (including flRAGE) isoforms and/or increase expression of the sRAGE (including RAGEv1) isoforms as screened in vivo in normal mice. Candidate SMOs are developed that target splicing of human RAGE pre-mRNA to reduce expression of the flRAGE isoform and/or increase expression of the RAGEv1 isoform. A set of molecular engineering tools are used to identify ranked panels of SMOs that can be used to decrease the expression of the flRAGE isoform and/or increase expression of the RAGEv1 isoform. These SMOs are then tested and the process is refined iteratively from those sequences which provide at least 5% alteration of flRAGE isoform and/or increase expression of the RAGEv1 isoform expression to select the most potent SMO candidates for further testing.

SMOs are developed to facilitate specific alternative inclusion of all or part of intron 9, which includes a stop codon, in the coding sequence, resulting in reduced expression of the membrane bound RAGE (including flRAGE) protein and/or increased expression of sRAGE (including RAGEv1) protein. Critical splicing motifs are predicted in silico using the most advanced RNA and oligo analysis tools. SMOs targeting RAGE alternative splicing is designed to target either the 3′ or 5′ splice sites and/or sequences corresponding to predicted ESE/ISE clusters near the splice junctions of exon 9 and intron 9. The following summarizes the SMO design process.

First, conservation between human and mouse RAGE sequences is identified. Alignments of the AGER gene sequences across species have been performed to determine conservation between mouse and human. Although similar, the alternative splicing event that produces RAGEv1 differs between mice and humans to the extent that we have developed transgenic mice with the full human AGER gene encoding for the RAGE protein for in vivo efficacy screening. Using these animals, SMOs developed and tested in mice can be translated directly to human use.

Second, ESE/ESS/ISE/ISS motifs surrounding the 3′ and 5′ splice sites of alternatively spliced exons in RAGE pre-mRNA are identified. RAGE exon 9 and intron 9 pre-mRNA sequences were surveyed for possible human spicing regulatory motifs. Possible ESE motifs were defined using ESE Finder (Cartegni et al., Nucl. Acids Res. 31(13):3568-3571, 2003), RESCUE-ESE (Dravet et al., Epilepsia 52 Suppl 2 (1-2), 2011; Fairbrother et al., Science 297(5583):1007-1013, 2002), and PESX (Zhang et al., Genes Dev. 18(11):1241-1250, 2004). Possible ESS elements were identified by PESX, and the two hexamer data set analysis by FAS-ESS (Wang et al., Cell 119(6):831-845, 2004) tool. Finally, possible ISE motifs were determined using the ACESCAN2 application (Yeo et al., Proc. Natl. Acad. Sci. U.S.A. 102(8):2850-2855, 2005; Yeo et al., PLoS. Genet. 3(5):e85, 2007).

Third, RNA Structure and Thermodynamics of RAGE target sequences was assessed. The RNA Structure program (Mathews et al., Proc. Natl. Acad. Sci. U.S.A. 101(19):7287-7292, 2004) can be used to predict secondary structure of target sequences and thermodynamic properties of all potential SMOs targeting RAGE. Additionally, sequence motifs and structures known or predicted to cause immune stimulation or other toxicities, can be screened for and avoided.

Fourth, BLASTN analysis of potential off-target hybridization is carried out to screen all candidate SMOs for potential hybridization to off-target sites in the human/mouse genomes. SMOs with greater than 85-95% off-target hybridization to any other known gene product are not considered.

Fifth, SMOs are prioritized based on their combined properties. In particular, thermodynamic properties between SMOs and their target, and self-self binding energies of SMOs, splice site strength, and possible splicing motifs are combined to establish top candidate SMOs for empirical screening and evaluation of splicing specificity and efficiency. These parameters used to select top candidate SMOs for initial screening are all contained in the above referenced oligonucleotide and RNA structure predictive software.

### In Vivo Splicing Efficacy

The U87-MG and SYSY lines (authenticated by STR DNA fingerprinting analysis—ATCC, USA) are human glioblastoma and neuroblastoma cells, respectively, that endogenously express RAGE (Leclerc et al., J. Biol. Chem. 282(43):31317-31331, 2007). Briefly, cell lines are maintained in RPMI medium containing 10% fetal bovine serum, 2 mM glutamine and streptomycin/penicillin (Leclerc et al., supra). Cells are plated and grown for 1 week or until they reach 50% confluence. RAGE SMOs are then complexed with oligofectamine, applied to each cell line (250 μM SMO), and incubated in reduced serum medium for 24 hours. Medium is replaced and cells grown for an additional 24-48 hours and harvested. GluA3-flip is also endogenously expressed in SYSY cells (Christnacher et al., FEBS Lett. 373(1):93-96, 1995), thus vehicle is used as a negative control, and LSP-GR3 (that reduces expression of GluA3-flip mRNA) as a positive control. Cell viability and cytotoxicity assessment is performed using Alamar Blue (Hamid et al., Toxicol. In Vitro 18(5):703-710, 2004). For mbRAGE and sRAGE mRNA expression, cells are lysed, total RNA extracted (Trizol), converted to cDNA (Mulitscribe reverse transcriptase kit; Applied Biosystems), and examined by real-time quantitative PCR (RT-QPCR). The level of mbRAGE transcripts are measured by TaqMan Gene Expression Assay Hs00542592_g1 (ThermoFisher Scientific). For quantification of sRAGE isoforms, a custom primer and probe set is designed using Primer Express (Applied Biosystems) and is validated for efficiency over 5 logs of cDNA concentration. Relative transcript expression is evaluated by the ΔΔCT method (Livak et al., Methods 25:402-408, 2001) relative to the geometric mean of β-Actin and TBP as endogenous control (Kreth et al., Neuro. Oncol. 12(6):570-579, 2010).

Using an iterative process of SMO evaluation and optimization, we analyze the efficacy of the 5 top-ranked SMOs, and use these data to make our next SMO choices in a strategic manner. For example, if an SMO shows a significant but incomplete reduction in flRAGE expression, bases may be added or subtracted from either end of the SMO sequence, to further improve efficacy. Although complete reduction of flRAGE expression may not be desirable, increased SMO potency will increase the therapeutic index. SMOs that show the greatest decrease in flRAGE and increase in RAGEv1 isoform expression are evaluated for dose-response over concentrations ranging from 0-250 μM.

### Determination of In Vivo Pre-mRNA Splicing and SMO Efficacy

Investigation of sRAGE alternative splicing variants revealed orthologous isoforms resulting from the partial inclusion of intron 9 and removal of exon 10 across several relevant species, including monkey, cow, rat, and mouse (Lopez-Diez et al., Genome Biol. Evol. 5(12):2420-2435, 2013). However, alignment of the genomic sequence of human and mouse RAGE revealed distinct differences in the human and mouse sRAGE isoforms, including: 1. stop codon for mouse sRAGE occurs earlier in the transcript, yielding a shorter truncated protein (EGLD; SEQ ID NO: 2901) compared to human (EGFDKVREAEDSPQHM; SEQ ID NO: 2902), 2. the alternative donor sites in intron 9 and the alternative acceptor sites in intron 9 (human) and exon 10 (mouse) are not 100% conserved between species (Kalea et al., FASEB J. 23(6):1766-1774, 2009). Thus, insertion of the human RAGE transgene into mouse is the most efficient method of directly testing in vivo efficacy for both target engagement and efficacy in animal models of disease such that, SMOs developed in cell culture can be tested in mice with resulting data directly translatable to clinical use. Although mouse models have been developed to express RAGE transgenes (Arancio et al., EMBO J. 23(20):4096-4105, 2004; Cho et al., FASEB J. 23(8):2639-2649, 2009) none express transgenes capable of alternative splicing in the CNS.

### Transgenic Human RAGE Expression in Mouse

Transgenic mouse generation was accomplished through constructing a large fragment nuclease expression vector containing the 1.49 kb human RAGE gene which was delivered via CRISPR/Cas-9 to obtain founder mice on a C57/B6 background. A PCR genotyping assay was developed to identify founders and offspring carrying the transgene. RAGE transgene expression in liver, kidney, and brain tissues is verified by RT-QPCR. Additional details regarding the production of these mice are provided above.

### In Vivo SMO Validation of Target Engagement for Lead Selection

Efficacy of top candidate SMOs is evaluated in neonatal RAGE transgenic (Tg) C57/B16 pups. SMOs undergo further iterative evaluation and optimization in vivo, where splicing efficacy of the top ranked SMO is examined and the results used to strategically select better optimized versions of that SMOs, as necessary. RAGE transgenic mice are given bilateral ICV injections of SMO (4 μg per lateral ventricle) on post-natal (P)3, P5, and P10, with brain tissues harvested at P12 and processed as previously described (Williams et al., J. Neurosci. 29 (24):7633-7638, 2009; Lykens et al., PLoS One. 2017 Feb. 8; 12(2):e0171538). RAGE expression is highest in the brain during development (Leclerc et al., J. Biol. Chem. 282 (43), 31317-31331, 2007), particularly in the hippocampus, cortical neurons, and glia (Malherbe et al., Brain Res. Mol. Brain Res. 71(2):159-170, 1999). Expression of membrane bound RAGE (including flRAGE) and sRAGE (including RAGEv1) is evaluated by RT-QPCR, and total sRAGE assessed in plasma and CSF from RAGE Tg mice by ELISA (BioVendor, Czech Republic).

Alternatively, mouse-specific SMOs corresponding to those candidates identified in the human cell culture screening are developed for validation of in vivo SMO effect on target. These mouse-specific SMOs are then used for all subsequent in vivo mouse studies. Mice express homologous RAGE isoforms compared to humans (Kalea et al., FASEB J. 23(6):1766-1774, 2009; Lopez-Diez et al., Genome Biol. Evol. 5(12):2420-2435, 2013) such that SMOs that specifically reduce membrane bound RAGE (including flRAGE) and sRAGE (including RAGEv1) isoforms in mouse can be used for proof-of-concept studies in disease models of AD. There is some controversy as to whether mice express detectable levels of sRAGE protein (Kalea et al., FASEB J. 23(6):1766-1774, 2009). However, RAGE SMOs identified in vitro are expected to produce the same effects on membrane bound RAGE (including flRAGE) and sRAGE (including RAGEv1) transcript expression in vivo in the transgenic mice.

### In Vivo Testing of RAGE SMOs in an Acute Model of Sporadic AD

Administration of ICY streptozotocin (STZ) is a well-characterized acute model of sporadic AD for early drug candidate screening causing acute cognitive deficits and neurodegeneration/inflammation, tau-hyperphosphorylation within 6 weeks after induction (Chen et al., Mol. Neurobiol. 47(2):711-725, 2013; Saxena et al., Pharmacol. Biochem. Behay. 86(4):797-805, 2007; Saxena et al., Eur. J. Pharmacol. 581(3):283-289, 2008; Grieb, Mol. Neurobiol. 53(3):1741-1752, 2016). Since ICV STZ is also associated with fatty liver, pancreatic islet hypertrophy, and related metabolic abnormalities known to contribute to AD (Bloch et al., J. Alzh. Dis., 60(1):121-136, 2017), we are assessing both ICV and subcutanteous (SC) administration of SMOs to alter brain and peripheral RAGE isoform expression. In non-human primate studies and clinical trials, SMOs delivered by lumbar intrathecal (i.t.) injection readily circulate to, and diffuse throughout the brain (Chiriboga et al., Neurology 86(10); 890-897, 2016; Geary et al., Adv. Drug Deliv. Rev. 87:46-51, 2015; Hache et al., J. Child Neurol. 2016; Finkel, Neurology (Meeting Abstracts) (P5.001), 2016; Rigo et al., J. Pharmacol. Exp. Ther. 350(1):46-55, 2014), without a need for invasive ventricular brain delivery. Although lumbar i.t. bolus injection may be the route of delivery for human clinical trials, the small i.t. space in mice makes consistent delivery to the brain technically challenging (Rigo et al., supra). However, direct ICV delivery is readily feasible in mice, thus, ICY dosing is proposed herein. Treated and control animals are litter-matched to reduce variability, and experimenters are blinded to treatment group.

### Determine the Dose-Response of Lead SMO(s) and Establish SC and ICV Dose Paradigms in Adult RAGE Transgenic Mice

Adult 129 mice at 6-8 weeks of age are implanted with a custom (Plastics One, Roanoke, Va.), bilateral cannula, which is inserted 1 mm lateral and 0.3 mm caudal to Bregma, and 3 mm in depth into each lateral ventricle, anchored to the skull for repeat ICV dosing access. After a minimum 1 week recovery, mice are given bilateral ICV injection of RAGE SMO or saline (5, 10, or 20 μg in 5 μL total volume), or aged matched mice are given SC injection (20, 40, or 80 μs) in the flank. Liver, kidney, and brain tissues are collected for RT-QPCR and Western blot or ELISA at 1 week and 4 weeks after the final dose. If SMO effect is not maintained out to 4 weeks post-dose, additional doses will be added. This experiment requires 6 treated mice (3M, 3F)/dose, at 6 doses, and 2 time-points, plus sets of ICV and SC vehicle controls at each time point for a total of 96 mice.

### SMO Efficacy Screening in an Acute Sporadic AD Model in RAGE Transgenic Mice

Two weeks prior to the start of studies, RAGE-Tg mice are cannulated in both lateral ventricles. Single SC and ICY doses are selected based on the described dose-response of lead SMO(s) to establish SC and ICV dose paradigms in adult RAGE transgenic 129 mice. This experiment requires 18 treated mice (9 male, 9 female)/treatment group to adequately power the study (Chen et al., Mol. Neurobiol. 47(2):711-725, 2013), with 4 treatments (SMO or vehicle given ICV or SC) and 2 time-points for a total of 144 mice.

a. Cognitive Testing:

Beginning at 8-10 weeks of age, mice are assessed cognitively for novel object recognition, Morris water maze, and passive avoidance tests, all prior to STZ induction (0.5 mg/kg in 10 μL total volume with two ICY doses 48 hours apart) and 2-3 weeks post-STZ injection as described previously (Chen et al., Mol. Neurobiol. 47(2):711-725, 2013; Saxena et al., Pharmacol. Biochem. Behay. 86(4):797-805, 2007; Saxena et al., Eur. J. Pharmacol. 581(3):283-289, 2008). Mice are dosed with vehicle control or SMO either 3 days prior to STZ administration or 24 hours post-STZ inductions. Behavior studies are performed using each mouse as its own baseline control as well as comparing to separate vehicle controls.

b. Histopathology:

At 6-8 weeks post-STZ induction mice are euthanized and brains sections examined for Tau hyper-phosphorylation at Ser199/202, Thr205, and Ser214 by Western Blot (Chen et al., Mol. Neurobiol. 47(2):711-725, 2013).

SMO treatment may alternatively be assessed by acute Aβ injection to model sporadic AD, with SMO effect on cognition assessed by modified hole board test (Schmid et al., Behay. Brain Res. 324:15-20, 2017).

### Statistical Analysis

Statistical calculations are performed using GraphPad or StatistiXL with significance set at p<0.05 and the mean±SEM determined for each treatment group. RT-Q PCR results are evaluated by student's t-test with Bonferoni correction for multiple comparisons when appropriate. For behavior tests, latency time comparisons among groups are performed by ANOVA followed by Tukey's post-hoc test.

### Other Embodiments

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

References to sequences in sequence databases herein, such as Genbank Accession numbers and the like, are intended to refer to those sequences as present in the respective databases on the date of filing of this application.

Embodiments of the invention are within the scope of the following numbered paragraphs.
1. A method of modulating splicing of a Receptor for Advanced Glycation End products (RAGE) pre-mRNA, the method comprising contacting a plurality of cells with a splice modulating oligonucleotide (SMO) that specifically binds to a complementary sequence of a pre-mRNA that undergoes splicing to form mRNA encoding a RAGE protein, wherein the SMO alters the relative amounts of mRNA encoding soluble and membrane bound isoforms of RAGE protein produced by the pre-mRNA splicing.
2. The method of paragraph 1, wherein the SMO increases the amount of mRNA encoding a soluble isoform of RAGE protein produced.
3. The method of paragraph 1 or 2, wherein the SMO decreases the amount of mRNA encoding a membrane bound isoform of RAGE protein.
4. The method of any one of paragraphs 1 to 3, wherein the SMO directs read-through of the 5′ splice site of exon 9 of the RAGE pre-mRNA, resulting in inclusion of part or all of intron 9, or exclusion of exon 10, or any combination thereof, in the RAGE pre-mRNA.
5. The method of any one of paragraphs 1 to 4, wherein the plurality of cells is in vitro.
6. The method of any one of paragraphs 1 to 5, wherein the plurality of cells is in vivo.
7. The method of any one of paragraphs 1 to 6, wherein the SMO specifically binds to a complementary sequence of RAGE pre-mRNA in at least one of the group consisting of an exon, an intron, a 5′ UTR, a 3′ UTR, a splice junction, an exon:exon splice junction, an exonic splicing silencer (ESS), an exonic splicing enhancer (ESE), an intronic splicing silencer (ISS), and/or an intronic splicing enhancer (ISE) or a combination of any of the aforementioned in the RAGE pre-mRNA.
8. The method of any one of paragraphs 1 to 7, wherein the SMO produces at least a 5 percent increase in read-through of the 5′ splice site of exon 9, resulting in inclusion of part or all of intron 9, or exclusion of exon 10, or any combination thereof, in a RAGE mRNA, as compared to baseline untreated cells, and alters expression of RAGE or one or more isoforms thereof.
9. The method of paragraph 6, wherein the plurality of cells is in vivo and the SMO is administered to a subject to treat a disease or condition selected from the group consisting of Alzheimer's disease, amyotrophic lateral sclerosis, diabetes, glucose tolerance, diabetic allodynia and neuropathy, diabetic retinopathy, atherosclerosis (e.g., coronary artery disease and peripheral artery disease), diabetic nephropathy, diabetic wound healing, cardiovascular disease, heart failure, ischemia-reperfusion injury, immunological disease, autoimmune disease (e.g., multiple sclerosis, osteoarthritis, and rheumatoid arthritis), sepsis, transplant rejection, cancer (e.g., glioma, breast cancer, liver cancer), pain, liver disease (e.g., hepatitis and liver fibrosis), and lung disease (e.g., acute airway injury and respiratory distress syndrome, chronic obstructive pulmonary disease, emphysema, asthma, cystic fibrosis, and idiopathic pulmonary fibrosis).
10. A splice modulating oligonucleotide (SMO) comprising 10 to 200 (e.g., 15 to 100, or 20 to 50) nucleotides that are complementary to an exonic or intronic sequence within exon 9, intron 9, or exon 10 of a RAGE pre-mRNA and an optional one or two additional nucleotides.
11. The SMO of paragraph 10, wherein the SMO sequence comprises one of SEQ ID NOs: 5-2897 or a variant thereof having at least 90% sequence identity to the reference sequence.
12. The SMO of paragraph 10 or 11, wherein the SMO sequence comprises one of SEQ ID NOs. 5-2897.
13. The SMO of any one of paragraphs 10 to 12, wherein at least one nucleotide in the SMO comprises a non-naturally occurring modification comprising at least one of a chemical composition of phosphorothioate 2′-O-methyl, phosphorothioate 2′-MOE, locked nucleic acid (LNA) including thiol-LNA, a constrained moiety, including a constrained ethyl nucleic acid (cEt) or constrained methoxyethyl (cMOE), peptide nucleic acid (PNA), phosphorodiamidate morpholino (PMO), cholesterol, GalNAc or any combination thereof.
14. The SMO of any one of paragraphs 10 to 13, wherein at least one of the nucleotides of the SMO is a phosphorothioate 2′-O-methyl modified nucleotide.
15. A pharmaceutical composition comprising an SMO of any one of paragraphs 10 to 14 and a pharmaceutically acceptable carrier or diluent.
16. A method of treating or preventing a disease or condition in a subject that would benefit from altered splicing of RAGE pre-mRNA, the method comprising administering to the subject an SMO of any one of paragraphs 10 to 14 or a composition of paragraph 15.
17. The method of paragraph 16, wherein the disease or condition is selected from the group consisting of Alzheimer's disease, amyotrophic lateral sclerosis, diabetes, glucose tolerance, diabetic allodynia and neuropathy, diabetic retinopathy, atherosclerosis (e.g., coronary artery disease and peripheral artery disease), diabetic nephropathy, diabetic wound healing, cardiovascular disease, heart failure, ischemia-reperfusion injury, immunological disease, autoimmune disease (e.g., multiple sclerosis, osteoarthritis, and rheumatoid arthritis), sepsis, transplant rejection, cancer (e.g., glioma, breast cancer, liver cancer), pain, liver disease (e.g., hepatitis and liver fibrosis), and lung disease (e.g., acute airway injury and respiratory distress syndrome, chronic obstructive pulmonary disease, emphysema, asthma, cystic fibrosis, and idiopathic pulmonary fibrosis).
18. The method of any one of paragraphs 1 to 9, wherein the SMO is an SMO of any one of paragraphs 10 to 14.
19. A non-human animal comprising a gene encoding human RAGE.
20. The non-human animal of paragraph 19, wherein the non-human animal is a mouse.
21. The non-human animal of paragraph 19 or 20, wherein the gene encoding human RAGE has been introduced into the genome of the non-human animal.
22. The non-human animal of any one of paragraphs 19 to 21, wherein the gene encoding RAGE of the non-human animal has been edited out, knocked out, and/or replaced with the gene encoding human RAGE.
23. The non-human animal of any one of paragraphs 19 to 22, wherein the gene encoding human RAGE is a genomic sequence, encoding exons and introns.
24. The non-human animal of any one of paragraphs 19 to 23, wherein the gene encoding human RAGE is under control of the endogenous RAGE promoter of the non-human animal.
25. The non-human animal of any one of paragraphs 19 to 24, wherein the non-human animal comprises a disease-related mutation.
26. The non-human animal of paragraph 25, wherein the disease-related mutation is in a gene encoding presenilin, SOD1, or the cystic fibrosis membrane transporter (CFTR).
27. The non-human animal of any one of paragraphs 19 to 26, which is an inducible disease model.
28. The non-human animal of paragraph 27, wherein the non-human animal is an inducible disease model of a disease selected from the group consisting of Alzheimer's disease, amyotrophic lateral sclerosis, diabetes, glucose tolerance, diabetic allodynia and neuropathy, diabetic retinopathy, atherosclerosis, diabetic nephropathy, diabetic wound healing, cardiovascular disease, heart failure, ischemia-reperfusion injury, immunological disease, autoimmune disease, sepsis, transplant rejection, cancer, pain, liver disease, and lung disease, and optionally effects on physiology or disease are assessed.
29. A method for identifying or characterizing an SMO directed against human RAGE pre-mRNA, the method comprising introducing an SMO into a non-human animal of any one of paragraphs 19 to 28 and assessing the effects of the SMO on the non-human animal.
30. The method of paragraph 29, wherein effects on splicing of RAGE pre-mRNA are assessed.
31. The method of paragraph 29 or 30, wherein the non-human animal is a disease model and a feature of the disease is assessed.
32. The method of any one of paragraphs 29 to 31, wherein the SMO comprises a sequence selected from SEQ ID NOs: 5-2897.

Other embodiments are within the scope of the following claims.

What is claimed is:

1. A method of modulating splicing of a Receptor for Advanced Glycation End products (RAGE) pre-mRNA, the method comprising contacting a plurality of cells with a splice modulating oligonucleotide (SMO) that specifically binds to a complementary sequence of a pre-mRNA that undergoes splicing to form mRNA encoding a RAGE protein, wherein the SMO alters the relative amounts of mRNA encoding soluble and membrane bound isoforms of RAGE protein produced by the pre-mRNA splicing.

2. The method of claim 1, wherein the SMO increases the amount of mRNA encoding a soluble isoform of RAGE protein produced.

3. The method of claim 1, wherein the SMO decreases the amount of mRNA encoding a membrane bound isoform of RAGE protein.

4. The method of claim 1, wherein the SMO directs read-through of the 5′ splice site of exon 9 of the RAGE pre-mRNA, resulting in inclusion of part or all of intron 9, or exclusion of exon 10, or any combination thereof, in the RAGE pre-mRNA.

5. The method of claim 1, wherein the plurality of cells is in vitro.

6. The method of claim 1, wherein the plurality of cells is in vivo.

7. The method of claim 1, wherein the SMO specifically binds to a complementary sequence of RAGE pre-mRNA in at least one of the group consisting of an exon, an intron, a 5′ UTR, a 3′ UTR, a splice junction, an exon:exon splice junction, an exonic splicing silencer (ESS), an exonic splicing enhancer (ESE), an intronic splicing silencer (ISS), and/or an intronic splicing enhancer (ISE) or a combination of any of the aforementioned in the RAGE pre-mRNA.

8. The method of claim 1, wherein the SMO produces at least a 5 percent increase in read-through of the 5′ splice site of exon 9, resulting in inclusion of part or all of intron 9, or exclusion of exon 10, or any combination thereof, in a RAGE mRNA, as compared to baseline untreated cells, and alters expression of RAGE or one or more isoforms thereof.

9. The method of claim 6, wherein the plurality of cells is in vivo and the SMO is administered to a subject to treat a disease or condition selected from the group consisting of Alzheimer's disease, amyotrophic lateral sclerosis, diabetes, glucose tolerance, diabetic allodynia and neuropathy, diabetic retinopathy, atherosclerosis (e.g., coronary artery disease and peripheral artery disease), diabetic nephropathy, diabetic wound healing, cardiovascular disease, heart failure, ischemia-reperfusion injury, immunological disease, autoimmune disease (e.g., multiple sclerosis, osteoarthritis, and rheumatoid arthritis), sepsis, transplant rejection, cancer (e.g., glioma, breast cancer, liver cancer), pain, liver disease (e.g., hepatitis and liver fibrosis), and lung disease (e.g., acute airway injury and respiratory distress syndrome, chronic obstructive pulmonary disease, emphysema, asthma, cystic fibrosis, and idiopathic pulmonary fibrosis).

10. A splice modulating oligonucleotide (SMO) comprising 10 to 200 nucleotides that are complementary to an exonic or intronic sequence within exon 9, intron 9, or exon 10 of a RAGE pre-mRNA and an optional one or two additional nucleotides.

11. The SMO of claim 10, wherein the SMO sequence comprises one of SEQ ID NOs: 5-2897 or a variant thereof having at least 90% sequence identity to the reference sequence.

12. The SMO of claim 10, wherein the SMO sequence comprises one of SEQ ID NOs. 5-2897.

13. The SMO of claim 10, wherein at least one nucleotide in the SMO comprises a non-naturally occurring modification comprising at least one of a chemical composition of phosphorothioate 2′-O-methyl, phosphorothioate 2′-MOE, locked nucleic acid (LNA) including thiol-LNA, a constrained moiety, including a constrained ethyl nucleic acid (cEt) or constrained methoxyethyl (cMOE), peptide nucleic acid (PNA), phosphorodiamidate morpholino (PMO), cholesterol, GalNAc or any combination thereof.

14. (canceled)

15. A pharmaceutical composition comprising an SMO of claim 10 and a pharmaceutically acceptable carrier or diluent.

16. A method of treating or preventing a disease or condition in a subject that would benefit from altered splicing of RAGE pre-mRNA, the method comprising administering to the subject an SMO of claim 10 or a pharmaceutical composition comprising the SMO.

17. The method of claim 16, wherein the disease or condition is selected from the group consisting of Alzheimer's disease, amyotrophic lateral sclerosis, diabetes, glucose tolerance, diabetic allodynia and neuropathy, diabetic retinopathy, atherosclerosis (e.g., coronary artery disease and peripheral artery disease), diabetic nephropathy, diabetic wound healing, cardiovascular disease, heart failure, ischemia-reperfusion injury, immunological disease, autoimmune disease (e.g., multiple sclerosis, osteoarthritis, and rheumatoid arthritis), sepsis, transplant rejection, cancer (e.g., glioma, breast cancer, liver cancer), pain, liver disease (e.g., hepatitis and liver fibrosis), and lung disease (e.g., acute airway injury and respiratory distress syndrome, chronic obstructive pulmonary disease, emphysema, asthma, cystic fibrosis, and idiopathic pulmonary fibrosis).

18. The method of claim 1, wherein the SMO is an SMO comprising 10 to 100 nucleotides that are complementary to an exonic or intronic sequence within exon 9, intron 9, or exon 10 of a RAGE pre-mRNA and an optional one or two additional nucleotides.

19. A non-human animal comprising a gene encoding human RAGE.

20. The non-human animal of claim 19, wherein:
(a) the non-human animal is a mouse;
(b) the gene encoding human RAGE has been introduced into the genome of the non-human animal;
(c) the gene encoding RAGE of the non-human animal has been edited out, knocked out, and/or replaced with the gene encoding human RAGE;
(d) the gene encoding human RAGE is a genomic sequence, encoding exons and introns;
(e) the gene encoding human RAGE is under control of the endogenous RAGE promoter of the non-human animal;
(f) the non-human animal comprises a disease-related mutation, which optionally is in a gene encoding presenilin, SOD1, or the cystic fibrosis membrane transporter (CFTR); or
(g) is an inducible disease model, which optionally is an inducible disease model of a disease selected from the group consisting of Alzheimer's disease, amyotrophic lateral sclerosis, diabetes, glucose tolerance, diabetic allodynia and neuropathy, diabetic retinopathy, atherosclerosis, diabetic nephropathy, diabetic wound healing, cardiovascular disease, heart failure, ischemia-reperfusion injury, immunological disease, autoimmune disease, sepsis, transplant rejection, cancer, pain, liver disease, and lung disease, and optionally effects on physiology or disease are assessed.

21-28. (canceled)

29. A method for identifying or characterizing an SMO directed against human RAGE pre-mRNA, the method comprising introducing an SMO into a non-human animal of claim 19 and assessing the effects of the SMO on the non-human animal, wherein optionally effects on splicing of RAGE pre-mRNA are assessed; the non-human animal is a disease model and a feature of the disease is assessed; or the SMO comprises a sequence selected from SEQ ID NOs: 5-2897.

30-32. (canceled)
